SEQUENCE LISTING (41 sequences)

SEQ ID NO: 1 - RNA, 22 nt, Artificial Sequence
  Description of Artificial Sequence Synthetic oligonucleotide
uuuguucguucggcucgcguga

SEQ ID NO: 2 - RNA, 22 nt, Artificial Sequence
  Description of Artificial Sequence Synthetic oligonucleotide
caaucacuaacuccacugccau

SEQ ID NO: 3 - RNA, 100 nt, Artificial Sequence
  Description of Artificial Sequence Synthetic polynucleotide
ugugucucucucuguguccugccagugguuuuacccuaugguagguuacgucaugcuguucuaccacaggguagaaccacggacaggauaccggggcacc

SEQ ID NO: 4 - RNA, 22 nt, Artificial Sequence
  Description of Artificial Sequence Synthetic oligonucleotide
ugagguaggagguuguauaguu

SEQ ID NO: 5 - RNA, 22 nt, Artificial Sequence
  Description of Artificial Sequence Synthetic oligonucleotide
uagcuuaucagacugauguuga

SEQ ID NO: 6 - DNA, 20 bp, Artificial Sequence
  Description of Artificial Sequence Synthetic primer
ggtggctgggaaaggagggg

SEQ ID NO: 7 - DNA, 30 bp, Artificial Sequence
  Description of Artificial Sequence Synthetic primer
ggctggtgctgagaggccgcccctgcctca

SEQ ID NO: 8 - DNA, 19 bp, Artificial Sequence
  Description of Artificial Sequence Synthetic primer
agggtggttgggaaaggag

SEQ ID NO: 9 - DNA, 22 bp, Artificial Sequence
  Description of Artificial Sequence Synthetic primer
ctaaaaaaccgcccctacctca

SEQ ID NO: 10 - DNA, 22 bp, Artificial Sequence
  Description of Artificial Sequence Synthetic primer
gtgttagtygtagatgagttta

SEQ ID NO: 11 - DNA, 20 bp, Artificial Sequence
  Description of Artificial Sequence Synthetic primer
gattaggattaggagattat

SEQ ID NO: 12 - DNA, 20 bp, Artificial Sequence
  Description of Artificial Sequence Synthetic primer
gatgagttttttgtataaat

SEQ ID NO: 13 - DNA, 29 bp, Artificial Sequence
  Description of Artificial Sequence Synthetic primer
gggggtagtttagaattagttggtgtttg

SEQ ID NO: 14 - DNA, 24 bp, Artificial Sequence
  Description of Artificial Sequence Synthetic primer
ccccttcttttcctttaccttccc

SEQ ID NO: 15 - DNA, 20 bp, Artificial Sequence
  Description of Artificial Sequence Synthetic primer
ggaaaggggagaagtgtggg

SEQ ID NO: 16 - DNA, 25 bp, Artificial Sequence
  Description of Artificial Sequence Synthetic primer
caccgcgtaccttaattatataaac

SEQ ID NO: 17 - DNA, 24 bp, Artificial Sequence
  Description of Artificial Sequence Synthetic primer
gaatttgggtttttattttttagg

SEQ ID NO: 18 - DNA, 24 bp, Artificial Sequence
  Description of Artificial Sequence Synthetic primer
ccaaaccctaaaactaactctctc

SEQ ID NO: 19 - DNA, 24 bp, Artificial Sequence
  Description of Artificial Sequence Synthetic primer
ctgcacctacccacatgcactcgt

SEQ ID NO: 20 - DNA, 26 bp, Artificial Sequence
  Description of Artificial Sequence Synthetic primer
ttaactagtactgggagggggaaggg

SEQ ID NO: 21 - DNA, 25 bp, Artificial Sequence
  Description of Artificial Sequence Synthetic primer
ggctgacagagaagattcccgagag

SEQ ID NO: 22 - DNA, 29 bp, Artificial Sequence
  Description of Artificial Sequence Synthetic primer
ccagtttagatggatcttggcagagagac

SEQ ID NO: 23 - DNA, 22 bp, Artificial Sequence
  Description of Artificial Sequence Synthetic primer
ttaccgcttcgtgcgctttgac

SEQ ID NO: 24 - DNA, 21 bp, Artificial Sequence
  Description of Artificial Sequence Synthetic primer
aacagcgtgcccctggacttg

SEQ ID NO: 25 - DNA, 21 bp, Artificial Sequence
  Description of Artificial Sequence Synthetic oligonucleotide
gtgctcggtttgtaggcagtg

SEQ ID NO: 26 - DNA, 22 bp, Artificial Sequence
  Description of Artificial Sequence Synthetic oligonucleotide
gtgccttgttttgatggcagtg

SEQ ID NO: 27 - DNA, 16 bp, Artificial Sequence
  Description of Artificial Sequence Synthetic oligonucleotide
ccccgcgacgagcccc

SEQ ID NO: 28 - DNA, 19 bp, Artificial Sequence
  Description of Artificial Sequence Synthetic oligonucleotide
gcctcacgcgagccgaacg

SEQ ID NO: 29 - DNA, 33 bp, Artificial Sequence
  Description of Artificial Sequence Synthetic primer
gggtagataaggagtagatttaattgattttag

SEQ ID NO: 30 - DNA, 30 bp, Artificial Sequence
  Description of Artificial Sequence Synthetic primer
ctctaaaccactcctaactcctaattactc

SEQ ID NO: 31 - DNA, 25 bp, Artificial Sequence
  Description of Artificial Sequence Synthetic primer
ggagttgattttttggagaaatggt

SEQ ID NO: 32 - DNA, 30 bp, Artificial Sequence
  Description of Artificial Sequence Synthetic primer
cttaccctaaacaaaaatcccaattccttc

SEQ ID NO: 33 - DNA, 31 bp, Artificial Sequence
  Description of Artificial Sequence Synthetic primer
gggtttagaagtttgtgtttgttatttaggg

SEQ ID NO: 34 - DNA, 29 bp, Artificial Sequence
  Description of Artificial Sequence Synthetic primer
cccaaaacaaaacgaccaccaacaacaac

SEQ ID NO: 35 - DNA, 33 bp, Artificial Sequence
  Description of Artificial Sequence Synthetic primer
gggttagtagttgatattttttgtttgttttag

SEQ ID NO: 36 - DNA, 35 bp, Artificial Sequence
  Description of Artificial Sequence Synthetic primer
cctaaaaaaaataataaccattttttatctaaccc

SEQ ID NO: 37 - DNA, 20 bp, Artificial Sequence
  Description of Artificial Sequence Synthetic primer
tgggaggaggggttgttgtt

SEQ ID NO: 38 - DNA, 25 bp, Artificial Sequence
  Description of Artificial Sequence Synthetic primer
taccaccctcccaaacacaaacttc

SEQ ID NO: 39 - DNA, 30 bp, Artificial Sequence
  Description of Artificial Sequence Synthetic primer
ggtggttatggtattaggagattatatggg

SEQ ID NO: 40 - DNA, 28 bp, Artificial Sequence
  Description of Artificial Sequence Synthetic primer
ctcttcccccaaaaaaaaaccaacatcc

SEQ ID NO: 41 - DNA, 278 bp, Artificial Sequence
  Description of Artificial Sequence Synthetic polynucleotide
ggctggtgctgagaggccgcccctgcctcacgcgagccgaacgaacaaaacgctcaggtccggtttgtgcgaggggctcgtcgcggggcgggagcggagggcccggggcctggcggtcgccctcggtgatctcctggtcctggtcttccgcgagctcagtgctgcacagcgccccgctagcctccgccctcacctgaacgcatctgcggctgacacgtcggcggccgcggcggccaccgcaggtgcgcacctggcccgcccctcctttcccagccacc

(19) United States
(12) Patent Application Publication
(10) Pub. No.: US 2013/0084328 A1
(43) Pub. Date: Apr. 4, 2013

(54) METHYLATED CODING AND NON-CODING RNA GENES AS DIAGNOSTIC AND THERAPEUTIC TOOLS FOR HUMAN MELANOMA

(71) Applicant: Ranjan Perera, Orlando, FL (US)

(72) Inventor: Ranjan Perera, Orlando, FL (US)

(73) Assignee: Sanford-Burnham Medical Research Institute

(21) Appl. No.: 13/457,350

(22) Filed: Apr. 26, 2012

Related U.S. Application Data
(60) Provisional application No. 61/479,766, filed on Apr. 27, 2011.
(60) Provisional application No. 61/479,791, filed on Apr. 27, 2011.

(51) Int. Cl. C12Q 1/68

(52) U.S. Cl. CPC C12Q 1/6869
USPC 424/450; 435/6.11; 435/6.12; 506/9; 514/44 A; 514/49

(57) ABSTRACT

Provided herein are methods for the diagnosis and treatment of human melanoma and prediction of early disease genesis to metastasis by assessing CpG island methylation or expression level of epigenetically regulated differentially expressed coding or non-coding genes. Methods of treatment of human melanoma by modifying or regulating the same pathways are also provided.

### CROSS-REFERENCE TO RELATED APPLICATIONS

The present application claims priority to and the benefit of U.S. Provisional Application Ser. Nos. 61/479,766, and 61/479,791, both of which were filed on Apr. 27, 2011, the specifications, drawings, claims, and abstracts of which are herein incorporated by reference in their entirety.

### STATEMENT OF GOVERNMENT-SPONSORED RESEARCH

This invention was made with United States government support awarded by the following agencies: National Institutes of Health under Grant No. 1R01GM084881-01, and the National Science Foundation under Grant No. FIBR 0527023. The United States government has certain rights in the invention.

### FIELD OF THE INVENTION

The present invention relates to methods of diagnosing and treating human melanoma.

### BACKGROUND OF THE INVENTION

The following discussion of the background of the invention is merely provided to aid the reader in understanding the invention and is not admitted to describe or constitute prior art to the present invention.

Melanoma, a cancer of pigment producing cells in the skin, has complex origins including genetic predisposition, prolonged exposure to ultraviolet rays of the sun, and the extent of melanin pigmentation of the skin. Melanoma is often highly metastatic and relatively resistant to standard chemotherapy (Nashan et al., 2007), and although less frequent than other types of skin cancers (such as basal or squamous cell carcinoma) it is the leading cause of death from skin cancers in human, and it is the leading cause of cancer-related death in females aged 20 to 35 years. The survival rate for advanced cases is less than 15% (2010 AJCC Melanoma Staging Database). One of the most frustrating and difficult areas in dermopathology and surgical pathology is the early and accurate diagnosis of cutaneous melanoma.

The exact molecular basis of melanoma initiation is uncertain. The primary mutagenic lesion may begin with DNA damage, followed by mutation in key tumor suppressor genes or resulting in aneuploidy. These initial changes are thought to progress into further mutations or epigenetic changes. Epigenetic changes in regulatory genes are often observed in melanoma, as in most cancers. These changes include CpG methylation in 5′-upstream cis regulatory elements and chromatin protein modification signatures that are associated with reduced or increased expression. Such changes can cause misregulation of tumor suppressor or oncogene expression either directly or indirectly through other regulatory genes (Esteller, 2007; Schuebel et al., 2007; Suzuki et al., 2002). For example, in one study, CpG islands in the promoter regions of several tumor suppressor genes were reported to be consistently hypermethylated in malignant melanoma cells (Hoon et al., 2004).

A number of protein-coding genes have been identified as potential biomarkers and candidate drug targets for melanoma, but development of additional sensitive and specific early diagnostic molecular biomarkers may open new avenues of treatment and prevention. Several of these genes exhibit distinct expression signatures among a variety of malignant metastatic melanomas and their benign forms.

Melanoma is a complex and heterogeneous disease, yet there are a number of molecular changes that are frequently seen in most melanomas. Though global transcript analysis can identify unrecognized subtypes of cutaneous melanoma and predict experimentally verifiable phenotypic characteristics that may be of importance to disease progression, aberrant methylation of promoter CpG islands is involved in silencing of tumor suppressor genes and is considered a potential cancer biomarker associated with a number of melanoma subtypes. Methylation at CpG islands is involved in silencing of tumor suppressor genes and is considered a potential cancer biomarker associated with a number of melanoma subtypes. Methylation at CpG islands results directly from the activities of cytosine DNA methyltransferases. Three DNA methyltransferase genes, DNAMT1, DNAMT3A, and DNAMT3B, have been identified in mammals. To date, approximately 50 genes have been identified to be regulated, at least in part, by hypermethylation of CpG islands in their respective regulatory regions in human melanomas. Of these, RASSF1 is a hallmark gene of abnormal methylation in many cancers including uveal and metastatic melanoma, raising the possibility that the presence of a methylated RASSF1A promoter region may serve as a melanoma marker.

An additional layer of complexity is provided by the abnormal expression of non-coding RNAs, mainly miRNAs, are emerging as early prognostic markers and therapeutic targets for a variety of diseases, and which can exert important influence at the posttranscriptional level in cancer cells (Perera and Ray, 2007). Indeed, several miRNAs exhibit distinct expression signatures among a variety of malignant metastatic melanomas and their benign forms. (Hoek et al., 2004). Since miRNA precursor genes are usually nested within other protein coding genes, often within intronic sequences, misregulation of these protein-coding genes by epigenetic mechanisms are expected to also cause aberrant regulation of miRNA target genes. miRNA gene silencing by CpG island methylation has been reported in several cancers (Han et al., 2007; Lujambio et al., 2007; Saito et al., 2006), though little is known for melanomas (Lujambio et al., 2008).

A number of miRNAs are known to be differentially expressed in melanoma, and several of the over-expressed miRNAs appear to regulate melanoma cell invasiveness and transcription and translation of tumor suppressor genes and oncogenes (Ma et al., 2009; Mueller and Bosserhoff, 2009; Mueller et al., 2009; Philippidou et al., 2010; Segura et al., 2010; Stark et al., 2010; Chen, 2005; Dalmay and Edwards, 2006; Esquela-Kerscher and Slack, 2006; Perera and Ray, 2007; Kent and Mendell, 2006; Zhang et al, 2006; Hammond, 2006). The miRNAs miR-221/222 down-regulate p27Kip1/CDKN1B and the c-KIT receptor mRNA levels, thereby controlling the progression of neoplasia, leading to enhanced proliferation and reduced differentiation in melanoma cells (Felicetti et al., 2008). miR-137 down-regulates the expression of MITF, a master regulator of cell growth, maturation, and pigmentation in melanoma cells (Bemis et al., 2008). It has recently been shown that several miRNA genes are differentially regulated in melanoma cells and one such miRNA, miR-211, is consistently reduced in melanoma and capable of targeting KCNMA1 mRNA, encoding a potassium transporter (Mazar et al., 2010). Increased KCNMA1 expression in melanoma cells, a consequence of the down-regulation of its negative regulator miR-211, is associated with increased invasiveness and high proliferative rates of these cells.

Additionally, miR-375 hypermethylation has been reported to be epigenetically modified in breast cancer and gastric cancer (de Souza Rocha Simonini et al., 2010; Tsukamoto et al., 2010, respectively). In breast cancer, higher expression of miR-375 in ERα-positive breast cell lines is shown to influence cell proliferation and miR-375 overexpression was caused by loss of epigenetic marks including H3K9me2 and local DNA hypomethylation Inhibiting miR-375 in ERα-positive MCF-7 cells resulted in reduced ERα activation and cell proliferation (de Souza Rocha Simonini et al., 2010). In gastric cancer, miR-375 was the most downregulated miRNA and its ectopic expression in gastric carcinoma cells markedly reduced cell viability via the caspase-mediated apoptosis pathway. Further, expression of miR-375 inhibited expression of PDK1, a direct target of miR-375, followed by suppression of Akt phosphorylation (Tsukamoto et al., 2010).

### SUMMARY OF THE INVENTION

In one aspect, the present invention provides a method for diagnosing melanoma in a subject suspected of having melanoma comprising: assessing the level of DNA methylation in a 5′ upstream CpG island of an epigenetically regulated differentially expressed coding or non-coding gene in a biological sample obtained from the subject; and determining whether the assessed level indicates hypermethylation; wherein hypermethylation indicates that the subject has melanoma, and the absence of hypermethylation indicates that the subject does not have melanoma. In some embodiments, the gene may be selected from the group consisting of miR-375, miR-34b, TERC, c-Kit, QPCT, CYP1B1, and PCSK1. In further embodiments, the gene may be miR-375, or miR-34b. In some embodiments, the biological sample may comprise skin, skin epidermis, melanocytes, melanocytic nevi, keratinocytes, melanoma cells, or tissue from other organs if the cancer is metastatic. The melanoma cells may, in further embodiments, be classified as primary in situ, regional metastatic, nodular metastatic, or distant metastatic.

In further embodiments, the level of DNA methylation may be assessed in a variety of ways, such as using methylation-specific PCR (MSP) or bisulphate DNA sequencing, or, in still further embodiments, may comprise a HELP assay, restriction landmark genomic scanning, methylated DNA immunoprecipitation (MeDIP), or highly methylated CpG islands pulled down by methylbinding domain protein MBD2 (Methyl_MBD). Further, when the gene is miR-375, the CpG island may be located from −170 to +58 bp upstream of the gene and contain 32 CpG dinucleotides.

Another aspect of the present invention provides a method of diagnosing or confirming a pathological stage of melanoma in a subject, said pathological stage being selected from the group consisting of stage I, stage II, stage III, and stage IV, and said method comprising (i) assessing the level of DNA methylation in a 5′ upstream CpG island of an epigenetically regulated differentially expressed coding or non-coding gene in a biological sample obtained from the subject; and (ii) identifying the subject as having stage I melanoma when the CpG island is less than about 20% methylated; identifying the subject as having stage II melanoma when the CpG island is from about 20% to about 60% methylated; identifying the subject as having stage III melanoma when the CpG island is greater than about 60% to about 85% methylated; and identifying the subject as having stage IV melanoma when the CpG island is greater than about 85% methylated. In some embodiments, the gene may be selected from the group consisting of miR-375, miR-34b, TERC, c-Kit, QPCT, CYP1B1, and PCSK1. In further embodiments, the gene may be miR-375, or miR-34b.

In some embodiments, the biological sample may comprise skin, skin epidermis, melanocytes, melanocytic nevi, keratinocytes, melanoma cells or tissue from other organs if the cancer is metastatic. Such melanoma cells may be, in some embodiments, classified as primary in situ, regional metastatic, nodular metastatic, or distant metastatic. The level of DNA methylation may be assessed in a variety of ways, such as, in some embodiments, methylation-specific PCR (MSP) or bisulphate DNA sequencing, or, in still further embodiments, may comprise a HELP assay, restriction landmark genomic scanning, methylated DNA immunoprecipitation (MeDIP), or highly methylated CpG islands pulled down by methylbinding domain protein MBD2 (Methyl_MBD). Further, in embodiments where the gene is miR-375, the CpG island may be located from −170 to +58 bp upstream of miR-375 and contains 32 CpG dinucleotides.

In another aspect, the present invention provides a method for diagnosing melanoma in a subject suspected of having melanoma comprising (i) assessing the level of an epigenetically regulated differentially expressed coding or non-coding gene in a biological sample obtained from the subject; (ii) comparing the expression level of the gene in the sample to a reference expression level derived from the expression level of the gene in samples obtained from subjects diagnosed as not having melanoma; and (iii) identifying the subject as having melanoma when the expression level of the gene in the sample is greater than the reference expression level or identifying the subject as not having melanoma when the expression level of the gene in the sample is not greater than the reference expression level. In some embodiments, the gene may be selected from the group consisting of miR-375, miR-34b, TERC, c-Kit, QPCT, CYP1B1, and PCSK1. In further embodiments, the gene may be miR-375, or miR-34b.

In further embodiments, the biological sample may comprise skin, skin epidermis, melanocytes, melanocytic nevi, keratinocytes, melanoma cells, or tissue from other organs if the cancer is metastatic. Such melanoma cells may be, in some embodiments, classified as primary in situ, regional metastatic, nodular metastatic, or distant metastatic. The expression level of the gene may be assessed in a variety of ways, such as, in some embodiments, evaluating the amount of the gene's mRNA in the biological sample. Such evaluation may be accomplished using a variety of methods, such as, in some embodiments, comprise reverse transcriptase PCR (RT-PCR), array hybridization, wherein the array may comprise an immobilized nucleic acid probe that specifically hybridizes the gene's mRNA, the gene's cDNA, or complements thereof.

In yet another aspect, the present invention provides a method for treating a patient diagnosed as having melanoma comprising administering to the patient an effective amount of a therapeutic agent that increases expression of a gene selected from the group consisting of miR-375, miR-34b, TERC, c-Kit, QPCT, CYP1B1, and PCSK1. The expression may, in some embodiments, be increased by at least 10%, at least 20%, at least 30%, at least 40%, at least 50%, at least 60%, at least 70%, at least 80%, at least 90%, or at least 100%. The therapeutic agent may, in further embodiments, also act to decrease CpG island methylation.

The therapeutic agent may, in some embodiments, be an anti-sense nucleic acid, which may be encoded in a vector or viral vector, or may be contained within a liposome. The therapeutic agent may further comprise 5AzadC, which may, in still further embodiments, be administered in conjunction with 4-PBA.

In still another aspect, the present invention provides a method for determining the invasiveness of melanoma in a subject comprising (i) assessing the expression level of an epigenetically regulated differentially expressed coding or non-coding gene in a biological sample obtained from the subject; and (ii) determining the invasiveness of the melanoma, wherein a higher expression level of the gene in the sample indicates lower invasiveness and a lower expression level of the gene in the sample indicates greater invasiveness. The expression level may, in some embodiments, be assessed by evaluating the amount of the gene's mRNA in the melanoma sample. Such an evaluation may comprise reverse transcriptase PCR (RT-PCR) or, in further embodiments, array hybridization, wherein the array comprises an immobilized nucleic acid probe that specifically hybridizes the gene's mRNA, cDNA, or complements thereof. In some embodiments, the gene may be selected from the group consisting of miR-375, miR-34b, TERC, c-Kit, QPCT, CYP1B1, and PCSK1. In further embodiments, the gene may be miR-375, or miR-34b.

In yet another aspect, a method is provided for determining the metastatic potential of melanoma in a subject comprising (i) assessing the expression level of an epigenetically regulated differentially expressed coding or non-coding gene in a biological sample obtained from the subject; and (ii) determining the metastatic potential of the melanoma, wherein a higher expression level of the gene in the sample indicates a lower metastatic potential and a lower expression level of the gene in the sample indicates a greater metastatic potential. The expression level may, in some embodiments, be assessed by evaluating the amount of the gene's mRNA in the melanoma sample. Such an evaluation may comprise reverse transcriptase PCR (RT-PCR) or, in further embodiments, array hybridization, wherein the array comprises an immobilized nucleic acid probe that specifically hybridizes the gene's mRNA, cDNA, or complements thereof. In some embodiments, the gene may be selected from the group consisting of miR-375, miR-34b, TERC, c-Kit, QPCT, CYP1B1, and PCSK1. In further embodiments, the gene may be miR-375, or miR-34b.

In still another aspect, a method is provided for determining the prognosis of a patient diagnosed as having melanoma comprising (i) assessing the expression level of an epigenetically regulated differentially expressed coding or non-coding gene in a biological sample obtained from the subject; and (ii) determining the expression level of the gene in the melanoma sample relative to the expression level of the gene in a reference sample, wherein a lower expression level of the gene in the melanoma sample relative to the reference sample indicates a poor prognosis. The expression level may, in some embodiments, be assessed by evaluating the amount of the gene's mRNA in the melanoma sample. Such an evaluation may comprise reverse transcriptase PCR (RT-PCR) or, in further embodiments, array hybridization, wherein the array comprises an immobilized nucleic acid probe that specifically hybridizes the gene's mRNA, cDNA, or complements thereof. In some embodiments, the gene may be selected from the group consisting of miR-375, miR-34b, TERC, c-Kit, QPCT, CYP1B1, and PCSK1. In further embodiments, the gene may be miR-375, or miR-34b.

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof). Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “short interfering nucleic acid” or “siNA” refers to any nucleic acid molecule capable of down regulating (i.e., inhibiting) gene expression in a mammalian cells (preferably a human cell). siNA includes without limitation nucleic acid molecules that are capable of mediating sequence specific RNAi, for example short interfering RNA (siRNA), double-stranded RNA (dsRNA), micro-RNA (miRNA), and short hairpin RNA (shRNA).

As used herein, the term “5′ upstream CpG island” refers to a CpG site-rich genomic region located upstream of the coding or non-coding RNA sequence. For example, the 5′ upstream CpG island may refer to a region in the 1Kbp upstream of the miR-375 sequence, an exemplary portion of which is depicted in FIG. 9, an example of which can be found in the region from −170 bps to +58 bps upstream of miR-375. Alternatively, the 5′ upstream CpG island may refer to a CpG site-rich genomic region in the putative promoter region of −631 bps to −396 bps upstream of miR-34b.

As used herein, the term “increase in biological activity” refers to any measurable increase of any biological effect caused by an increase in the expression of a nucleic acid or protein. An increase in biological activity may often be measured by increased amounts of RNA (e.g., mRNA) or protein, or may be measured functionally.

As used herein, “miR-375” is a nucleic acid molecule coded for by the gene miR-375 which is inversely-oriented in human chromosome 2 and which may include ribonucleotides, deoxyribonucleotides, or modified nucleotides exemplified by UUU GUU CGU UCG GCU CGC GUG A (SEQ ID NO:1) or substantially identical sequences thereto. Although the nucleotide sequence of miR-375 is provided as RNA, it is recognized that the a miR-375 nucleic acid may contain DNA, a mixture of RNA and DNA, and/or incorporate one or more modified bases or base substitutes.

As used herein, “miR-34b” is a nucleic acid molecule including ribonucleotides, deoxyribonucleotides, or modified nucleotides exemplified by SEQ ID NO:2 (CAA UCA CUA ACU CCA CUG CCA U) or substantially identical sequences thereto. Although the nucleotide sequence of miR-34b is provided as RNA, it is recognized that the miR-34b nucleic acid may contain DNA, a mixture of DNA and RNA, and/or incorporate one or more modified bases or base substitutes.

As used herein, “miR-140” is a nucleic acid molecule including ribonucleotides, deoxyribonucleotides, or modified nucleotides exemplified by SEQ ID NO:3 (UGU GUC UCU CUC UGU GUC CUG CCA GUG GUU UUA CCC UAU GGU AGG UUA CGU CAU GCU GUU CUA CCA CAG GGU AGA ACC ACG GAC AGG AUA CCG GGG CAC C) or substantially identical sequences thereto. Although the nucleotide sequence of miR-140 is provided as RNA, it is recognized that the miR-140 nucleic acid may contain DNA, a mixture of RNA and DNA, and/or incorporate one or more modified bases or base substitutes.

As used herein, sequences that are “substantially identical” to each other have identical nucleotides at least at about 50% of aligned nucleotide positions, preferably at least at about 75%, 80%, 85%, 90%, or 95% of aligned nucleotide positions, and more preferably at least at about 99% of aligned nucleotide positions.

As used herein, the term “sense region” refers to a nucleotide sequence of a molecule complementary (partially or fully) to an antisense region of the molecule. Optionally, the sense strand of a molecule may also include additional nucleotides not complementary to the antisense region of the molecule.

As used herein, the term “antisense region” refers to a nucleotide sequence of a molecule complementary (partially or fully) to a target nucleic acid sequence. Optionally, the antisense strand of a molecule may include additional nucleotides not complementary to the sense region of the molecule.

As used herein, the term “duplex region” refers to the region in two complementary or substantially complementary oligonucleotides that form base pairs with one another that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and/or 3′ overhangs.

An “abasic nucleotide” conforms to the general requirements of a nucleotide in that it contains a ribose or deoxyribose sugar and a phosphate but, unlike a normal nucleotide, it lacks a base (i.e., lacks an adenine, guanine, thymine, cytosine, or uracil). Abasic deoxyribose moieties include, for example, abasic deoxyribose-3′-phosphate; 1,2-dideoxy-D-ribofuranose-3-phosphate; 1,4-anhydro-2-deoxy-D-ribitol-3-phosphate.

As used herein, the term “HELP assay” refers to HpaII tiny fragment enrichment by ligation-mediated PCR, which is an assay that compares representations generated by HpaII and by MspI digestion of the genome followed by ligation-mediated PCR. HpaII, which only digests 5′-CCGG-3′ sites when the cytosine in the central CG dinucleotide is unmethylated, is added to a sample prior to PCR, and MspI is added to control for PCR amplification difficulties.

As used herein, “restriction landmark genomic scanning” refers to a genome analysis method involving use of restriction enzymes specific to DNA modifications to visualize restriction sites. After addition of a set of restriction enzymes, the DNA is directly labeled with a radioactive isotope, for example, phosphorus-32. A two-dimensional electrophoresis process is then employed, yielding high-resolution results. The radioactive second-dimension gel is then allowed to expose a large sheet of film, which is then developed, resulting in an autoradiograph.

As used herein, “methylated-DNA immunoprecipitation” or “MeDIP” refers to any method involving isolation of methylated DNA fragments via an antibody raised against 5-methylcytosine (5mC), including MeDIP-chip, in which the purified fraction of methylated DNA can further be input to high-resolution DNA microarrays, or MeDIP-seq, in which the fraction further undergoes sequencing.

As used herein, use of “methylbinding domain protein,” or “MBD2 (Methyl_MBD)” to assess levels of methylation of DNA fragments refers to any method in which MBD captures methylated DNA using a methyl-CpG binding domain-based (MBD) protein to discover differentially methylated regions (DMRs) in cancer.

As used herein, “bisulphite DNA sequencing” refers to any method comprising the steps of sequencing bisulphite treatment of DNA to determine its pattern of methylation. After treatment of DNA with bisulphite, which converts cytosine to uracil but leaves 5-methylcytosine unaffected, analysis may be conducted using any known method, including but not limited to non-methylation specific PCR methods such as direct sequencing, pyrosequencing, or MS-SSCA, methylation specific PCR, or microarray-based methods.

As used herein, the term “inhibit”, “down-regulate”, or “reduce,” with respect to gene expression, means that the level of RNA molecules encoding one or more proteins or protein subunits (e.g., mRNA) is reduced below that observed in the absence of the inhibitor. Expression may be reduced by at least 90%, 80%, 70%, 60%, 50%, 40%, 30%, 20%, 10%, 5% or below the expression level observed in the absence of the inhibitor.

As used herein, the term “hypermethylate” or “hypermethylated” refers to an occurrence of an epigenetic modification of the number 5 carbon of a cytosine pyrimidine ring by the addition of a methyl group to a degree that represses transcription of the gene.

As used herein, the term “poor prognosis” refers to the prognosis determined for a patient having melanoma which is worse (i.e., has a less favorable outcome) than the prognosis for a reference patient or group of patients with the same disease. For example, a patient with a poor prognosis may be expected to exhibit increased tumor growth rate and/or tumor invasiveness, increased frequency or number of metastasis, reduced remission duration, and/or reduced survival time relative to reference patients (i.e., patients having melanoma without a poor prognosis).

As used herein, the term “primary in situ” refers to a pathological stage associated with stage 0 or stage I melanoma wherein malignant cells have not yet metastasized or invaded beyond the original disease or tumor site.

As used herein, the term “regional metastatic” refers to a pathological stage associated with stage II melanoma usually characterized by an increase in size of a primary tumor and regional metastasis of malignant cells.

As used herein, the term “nodular metastatic” refers to a pathological stage associated with stage III melanoma wherein malignant cells have metastasized to at least one lymph node.

As used herein, the term “distant metastatic” refers to a pathological stage associated with stage IV melanoma usually characterized by distant metastasis, elevated LDH levels, and decreased likelihood of survival are likely.

As used herein, the term “differentially expressed” refers to a gene, such as, for example, miR-375, miR-34b, CYP1B1, c-Kit, QPCT, PCSK1, or TERC, which has variant expression levels in melanoma cells and melanocytes, or between cells in any two of the following stages: melanocytes, primary in situ, regional metastatic, distant metastatic, and nodular metastatic.

### BRIEF DESCRIPTION OF THE FIGURES

FIG. 1(A) is an illustration of the phylogenetic cluster of differentially expressed miRNAs in melanoma cells after methylation treatment; and FIG. 1(B) is a chart showing the fold changes in miRNA expression measured by qRT-PCR following methylation treatment.

FIG. 2 is a schematic representation of bisulfite genomic sequencing results showing frequency of CpG island methylation in the miRNA-375 upstream sequence.

FIG. 3 is a schematic representation of bisulfite genomic sequencing results showing CpG island methylation in the miRNA-375 putative promoter in patient samples.

FIG. 4(A) is a series of photomicrographs showing cell growth images of melanoma cell lines; FIG. 4(B) is a series of photomicrographs showing the cell morphology changes of the stained nucleus and cytoskeleton of melanoma cells ectopically expressing miR-375.

FIG. 5(A) is a line graph of the results of a cell proliferation assay; FIG. 5(B) is a series of photographs showing a wound healing assay with melanoma cells; FIG. 5(C) is a bar graph depicting the percent surface wound healing over time; FIG. 5(D) is a bar graph showing the results of a cell invasion assay; and FIG. 5(E) is a set of photographs showing the results of the cell invasion assay of FIG. 5(D).

FIG. 6 is a diagram showing the pathway mapping results of the top 20 downregulated miR-375 target genes in miR-375 ectopically expressed melanoma cells.

FIG. 7(A) is a representation of bisulfite genomic sequencing results showing frequency of CpG island methylation in cells treated with 5AzadC; and FIG. 7(B) represents northern blot analysis results detecting miR-375 levels after demethylation treatment.

FIG. 8 is a graph showing the top 20 putative miR-375 targets filtered by RPKM values and fold change differences from sequencing results.

FIG. 9 is a partial sequence (278 bp) of the inversely-oriented sequence upstream of miR-375 in human chromosome 2 containing one or more CpG islands. The pictured segment includes 190 bp of the upstream sequence and 88 bp internal to miR-375, and contains at least one CpG island.

FIG. 10 is a diagram depicting the workflow results of the DNA mapping to the human melanoma genome.

FIG. 11A is a Venn diagram demonstrating highly methylated CpG islands common and unique for melanocytes, WM793B and WM1552C. FIG. 11B is a Venn diagram showing the common and unique CpG islands between A375, HT-144, RPMI, and SKMEL-2 cells.

FIG. 12 is a line graph showing the global CpG island tag length and tag count in melanoma cell lines and melanocytes.

FIG. 13 is a line graph showing the percentage of methylation in genomics regions in melanoma cell lines and melanocytes.

FIG. 14A is a screen shot and an diagram showing the results of bisulphite sequencing of coding gene PCSK1 showing validation of promoter DNA methylation; FIG. 14B is a screen shot and an diagram showing the results of bisulphite sequencing of coding gene CYP1B1 showing validation of promoter DNA methylation; FIG. 14C is a screen shot and an diagram showing the results of bisulphite sequencing of coding gene QPCT showing validation of promoter DNA methylation; FIG. 14D is a screen shot and an diagram showing the results of bisulphite sequencing of coding gene c-Kit showing validation of promoter DNA methylation; and FIG. 14E is a screen shot and an diagram showing the results of bisulphite sequencing of coding gene TERC showing validation of promoter DNA methylation.

FIG. 15A is a representation of bisulfite genomic sequencing results showing frequency of CpG island methylation in the miRNA-34b upstream sequence, and (B) is a photograph of the northern blot results of melanoma cell lines for expression of miR-36b.

FIG. 16A is a representation of bisulfite genomic sequencing results showing CpG island methylation of the miRNA-34b putative promoter, and (B) is a photograph of the northern blot results showing expression of miR34 b after methylation.

FIG. 17 is a representation of bisulfite genomic sequencing results showing CpG island methylation in the miRNA putative promoter in patient samples.

FIG. 18(A) is a bar graph showing global expression profiling in melanoma cells; (B) is a bar graph demonstrating the most up- or down-regulated coding genes in melanoma cells; (C) is a bar graph demonstrating the most up-regulated miRNAs in melanoma cells; and (D) is a series of bar graphs providing qRT-PCR validation of several ORFs and miRNAs.

FIG. 19(A) is a heat map of top potential miR-34b targets; (B) is a pathway map of the top potential miR-34b targets; and (C) is a series of bar graphs showing qRT-PCR validation of two putative miR-34b targets.

FIG. 20(A) is a line graph showing a cell proliferation assay; (B) is a bar graph showing results of a cell adherence assay; (C) and (D) are bar graphs showing results of cell invasion assays; and (E) is a series of photographs showing results of cell invasion assays.

FIG. 21(A) is a series of photographs of cells immediately after scratch formation in a wound healing assay; and (B) is a bar graph showing a quantization of the results of the wound healing assay.

FIGS. 22(A) and (B) are depictions of the results of bisulfite genomic sequencing showing lack of methylation of CpG islands of miR-124-3 and miR-let-7i, respectively.

### DETAILED DESCRIPTION

The present invention is directed to the use of biomarkers in diagnosis and treatment of human melanoma. A biomarker indicative of melanoma can be determined in a biological sample. A biological sample may include a biopsy, a skin biopsy, a mole or nevus biopsy, blood, serum, cultured cells including primary and secondary (i.e. immortalized) cultured cells, or any combination thereof.

The discovery of the present invention was facilitated by a detailed characterization of epigenetic regulation of coding and non-coding genes in human melanoma conducted by the inventors, including the discovery that several genes in particular are highly methylated in advanced melanoma cell lines and patient samples, but not in melanocytic nevi, melanocytes, keratinocytes, or normal skin. As reported herein, a group of epigenetically regulated miRNA genes in melanoma cells were identified, and, through use of direct DNA bisulphate sequencing, it was confirmed that the upstream CpG islands of miR-375, miR-34b, TERC, c-Kit, QPCT, CYP1B1, and PCSK1 are hypermethylated in cell lines derived from different stages of melanoma, but not in control cells. Abnormalities in CpG island methylation were also seen in distinct melanoma patient samples classified as primary in situ, regional metastatic, nodular metastatic, and distant metastatic.

As shown in more detail in the working examples, the cell line WM1552C, derived from a stage 3 melanoma, was engineered to artificially express several genes, including miR-34b and miR-375. This caused a reduction in cell invasion, proliferation, and wound healing rates, consistent with the conclusion that reduced expression of these genes is related to invasiveness and motility of skin cancer melanoma. RNA samples isolated from WM1552C with or without gene expression were subjected to deep-sequencing (RNA_seq) to identify gene networks around the genes of interest. Network modules potentially regulated by the genes of interest are regulated to cytoskeletal remodeling and cell invasion, indicating a mechanism for the role of epigenetically regulated genes in regulating normal cell motility and morphology.

Provided herein are methods for diagnosing melanoma in a subject suspected of having melanoma or for determining the risk of a subject for developing melanoma by: (i) assessing the level of DNA methylation in a 5′ upstream CpG island of an epigenetically regulated differentially expressed coding or non-coding gene in a biological sample obtained from the subject; and (ii) identifying the subject as having or at risk of having melanoma when the sequences are hypermethylated or identifying the subject as not having or at risk of having melanoma when the sequences are not hypermethylated. Suitable biological samples include, but are not limited to, skin, skin epidermis, or melanocytes.

Methods for determining DNA methylation are known in the art. Suitable methods include methylation-specific PCR (MSP), a HELP assay, restriction landmark genomic scanning, or methylated DNA immunoprecipitation (MeDIP).

Provided herein are methods for diagnosing melanoma in a subject by: (i) assessing the level of an epigenetically regulated differentially expressed coding or non-coding gene in a biological sample obtained from the subject; (ii) comparing the expression level of the gene in the sample to the a reference expression level derived from the expression level of the gene in samples obtained from subjects diagnosed as not having melanoma; and (iii) identifying the subject as having melanoma when the expression level of the gene in the sample is greater than the reference expression level or identifying the subject as not having melanoma when the expression level of the gene in the sample is not greater than the reference expression level. Suitable biological samples include, but are not limited to, skin, skin epidermis, or melanocytes.

The expression level of the gene may be assessed by evaluating the amount of its mRNA in the biological sample. Methods for evaluating the amount of nucleic acid are known in the art and include, but art not limited to, reverse transcriptase PCR (RT-PCR) or array hybridization, wherein the array comprises an immobilized nucleic acid probe that specifically hybridizes the gene's mRNA, cDNA, or complements thereof.

Also provided herein are methods of diagnosing or confirming a pathological stage of melanoma in a subject, such as stage I, stage II, stage III, or stage IV, comprising (i) assessing the level of DNA methylation in a CpG island of an epigenetically regulated differentially expressed coding or non-coding gene in a biological sample as described above, and (ii) identifying the subject as having stage I melanoma when the CpG island is less than about 30% methylated; identifying the subject as having stage II melanoma when the CpG island is from about 30% to about 60% methylated; identifying the subject as having stage III melanoma when the CpG island is greater than about 60% to about 85% methylated; and identifying the subject as having stage IV melanoma when the CpG island is greater than about 85% methylated.

### MicroRNAs as Melanoma Biomarkers

Biomarkers useful for identifying subjects at risk for or for monitoring the progression of melanoma include non-coding RNAs (ncRNA) such as microRNAs. Quantification of one or more of these ncRNAs can indicate risk or progression of melanoma for the subject, and thus may provide opportunities for preventative treatments.

MicroRNAs are small, non-coding RNAs with an average length of about twenty-one to twenty-three base pairs. Though hundreds of miRNAs have been discovered in a variety of organisms, little is known about their cellular function. They have been implicated, for example, in post-transcriptional regulation, regulation of developmental timing and pattern formation, restriction of differentiation potential, regulation of insulin secretion, resistance to viral infection, and in genomic rearrangements associated with carcinogenesis and other genetic disorders, such as fragile X syndrome. While miRNAs have been linked to post-transcriptional and developmental regulations, other non-coding RNAs have little or no known function.

Recent evidence suggests that the number of unique miRNAs in humans alone could exceed 800, and may even be as high as 20,000. These post-transcriptional regulators of gene expression in higher eukaryotes play an important role in development, tumor suppression, and other cellular processes by hybridizing to complementary target messenger RNA (mRNA) transcripts and ultimately down-regulating or up-regulating gene expression depending on the abundance of that particular miRNA. Because of this unique function, special attention has been given to miRNAs as candidate drug targets for cancer, diabetes, obesity, and viral diseases, wherein miRNAs influence cancer development by serving as either tumor suppressors or oncogenes, but the regulation of miRNA is poorly investigated.

MicroRNAs have, however, joined the ranks of important regulatory molecules known to control human cell fate in normal and diseased states. This is true in many cancer cells, including melanoma cells, which are known to undergo progressive changes during disease manifestation. However, the extent and the importance of epigenetic regulation of miRNA expression in human melanoma cells have not been extensively investigated. A group of epigenetically regulated miRNA genes has been identified in melanoma cells, including miR-34b, -489, -375, -132, -142-3p, -200a, -145, -452, -21, -34c, -496, -let7e, -654, and -519b. Employing direct DNA bisulphite and immunoprecipitated methylated DNA (methyl-DIP) deep-sequencing it is confirmed that the upstream CpG island sequences of one such miRNA gene (miR-34b), is hypermethylated in cell lines derived from stages 3 and 4 metastatic melanoma, which correlate with low expression levels of this miRNA species in these cell lines. CpG methylation is less extensive in stage 1 and 2 melanoma samples, or in normal melanocytes and keratinocytes. Abnormalities in CpG island methylation also occur in distinct melanoma patient samples classified as primary in situ, regional metastatic and distant metastatic, though not in nodal metastatic samples.

Without wishing to be bound by any theory, it is proposed that during melanoma formation, initial genetic or epigenetic changes progress into additional mutations or further epigenetic changes, leading to abnormal functioning of several signaling pathways. Abnormal DNA methylation patterns at the 5′ noncoding region of the INK4a gene was discovered in melanoma (Jonsson et al., 2010), suggesting the involvement of epigenetic factors in melanoma development or progression. Epigenetic silencing of PTEN expression occurs in certain malignant melanomas with no detectable mutation in the PTEN gene (Zhou et al., 2000). While the impact on melanoma development of epigenetic changes in several protein coding genes is appreciated, few studies have reported on the impact of epigenetic regulation of non-coding RNAs, such as miRNAs.

A group of epigenetically regulated miRNA genes has been identified in melanoma cells, and by employing direct DNA bisulphite and immunoprecipitated methylated DNA (methyl-DIP) deep-sequencing, it has been confirmed that the upstream CpG island sequences of several such miRNA genes including miR-375 are hypermethylated in cell lines derived from different stages of melanoma but not in melanocytes and keratinocytes. Abnormalities in CpG island methylation were also shown in distinct melanoma patient samples classified as primary in situ, regional metastatic, and distant metastatic.

Epigenetic modification of DNA, such as methylation and/or histone modification, is thought to play a key role in cancer progression. In melanoma, epigenetic modifications may silence or reduce the expression of miRNAs, but their expression can be reactivated upon treatment with DNA methyltransferase inhibitors such as 5AzadC or histone deacetylating agents such as 4-PBA.

miR-375

The cell line WM1552C derived from a stage 3 melanoma was engineered to artificially express one such epigenetically modified miRNA, miR-375 (UUU GUU CGU UCG GCU CGC GUG A; SEQ ID NO:1). This caused a reduction in cell invasion, proliferation, and wound healing rates, and induced changes in cell morphology, suggesting that reduced expression of miR-375 is related to invasiveness, motility, and high cell division rate of WM1552C. RNA samples isolated from WM1552C with or without miR-375 expression were subjected to deep sequencing to identify gene networks around miR-375. Network modules potentially regulated by miR-375 are shown to be related to cytoskeletal remodeling and cell invasion, consistent with the conclusion that this mechanism provides a role for miR-375 in regulating normal cell motility and cytokinesis.

Treating melanoma-derived cell lines with 5-aza-2′-deoxycytidine (5-Aza-dC) markedly increases the expression levels of several miRNAs, suggesting that the corresponding genes encoding these miRNAs might be under the direct or indirect influence of epigenetic regulation by DNA methylation. DNA methylation of one such miRNA, miR-375 is shown herein to play a role in cell invasion and motility in human melanomas. Additionally, treatment of melanoma-derived cell lines with both 5AZAdC and also the histone deacetylase inhibitor 4-phenylbutyrate (4-PBA) increases the expression level of epigenetically regulated miRNAs still further, although treatment with 4-PBA alone had no statistically significant effects.

When these melanoma cell lines are engineered to express miR-375, they exhibit reduced proliferation rate, decreased substrate attachment, and reduced invasion and motility. miR-375 methylation was also seen in distinct melanoma patient samples, but to a much lesser extent in normal skin. This supports the idea that the epigenetic silencing of miR-375 is responsible for most of the cellular changes we report here through its protein coding target gene counterparts.

Therefore, these therapeutic properties render it highly desirable to formulate and administer pharmaceutical compositions comprising miR-375 nucleic acids or vectors as described in more detail below. An increase of biologically available miR-375 can, as described above, reduce proliferation, decrease substrate attachment, and reduce invasion and motility. Such an increase may be achieved directly, by introducing pharmaceutical compositions comprising miR-375 nucleic acids or vectors, or indirectly, by administration of a therapeutic agent which acts to upregulate the expression of the miR-375 gene. Upregulation as used herein refers to a process occurring in a cell triggered by an internal or external signal and resulting in increased expression of one or more genes.

The following general molecular biology techniques constitute examples of techniques that may be used in the present methods, but are not provided to constitute an exhaustive list or description.

miR-34b

The cell line WM1552C, derived from a stage 3 melanoma, was engineered to artificially express miR-34b. This caused a reduction in cell invasion, motility, attachment, and proliferation rates, suggesting that reduced expression of miR-34b is related to invasiveness and high cell division rate in WM1552C. RNA samples isolated from WM1552C with or without a miR-34b expression construct were subjected to deep sequencing to identify gene networks around miR-34b. These results demonstrate that network modules potentially regulated by miR-34b are related to cytoskeletal remodeling, and cell invasion, suggesting a mechanism for the role of miR-34b in regulating normal cell motility and cytokinesis.

When these melanoma cell lines are engineered to express miR-34b, they exhibit reduced proliferation rate, decreased substrate attachment, and reduced invasion and motility. miR-34b methylation was also seen in distinct melanoma patient samples, but to a much lesser extent in normal skin. This supports the idea that the epigenetic silencing of miR-34b is responsible for most of the cellular changes we report here through its protein coding target gene counterparts.

Provided herein are methods for diagnosing melanoma in a subject suspected of having melanoma or for determining the risk of a subject for developing melanoma by: (i) assessing the level of DNA methylation in a 5′ upstream CpG island of miR-34b in a biological sample obtained from the subject; and (ii) identifying the subject as having or at risk of having melanoma when the sequences are hypermethylated or identifying the subject as not having or at risk of having melanoma when the sequences are not hypermethylated. Suitable biological samples include, but are not limited to, skin, skin epidermis, or melanocytes.

### TERC

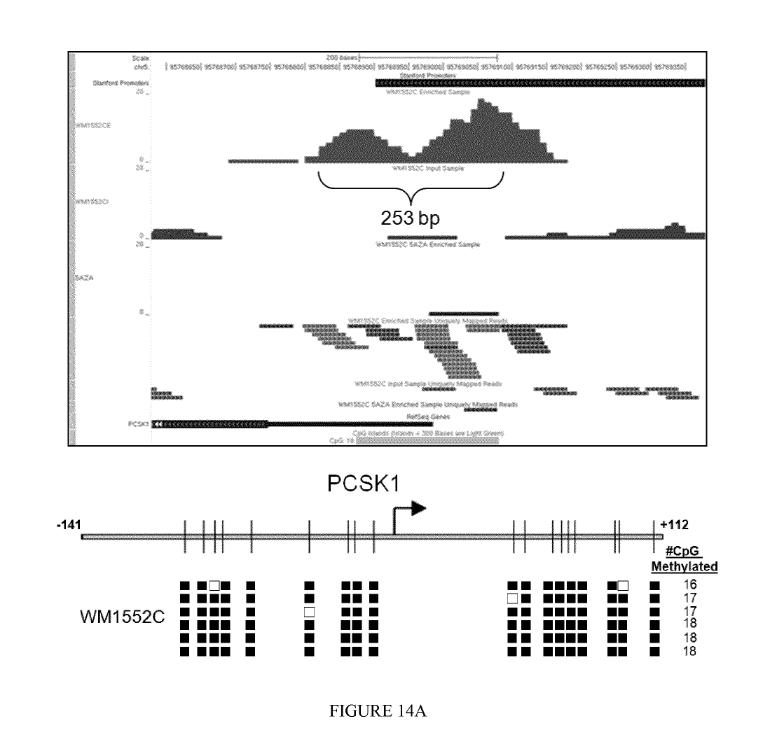

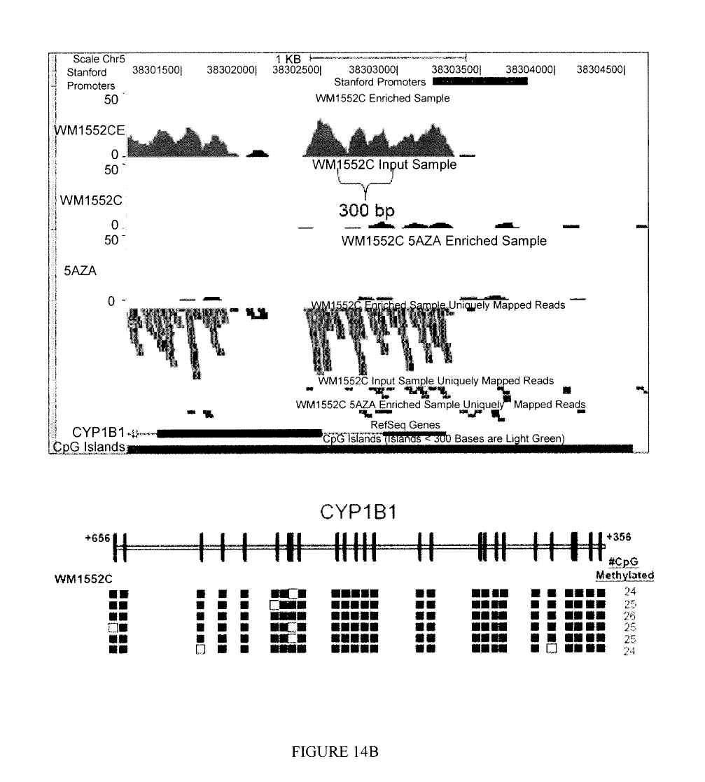

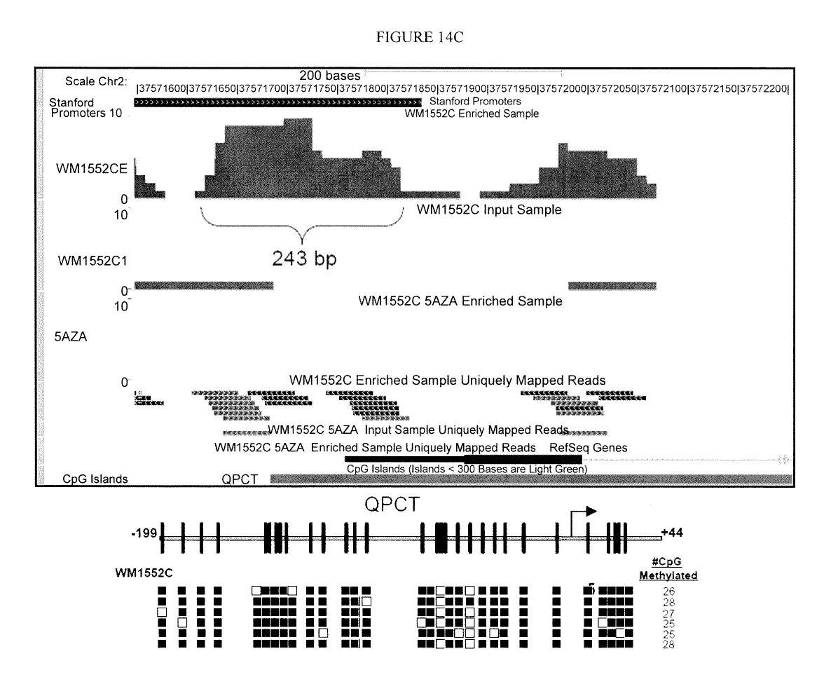

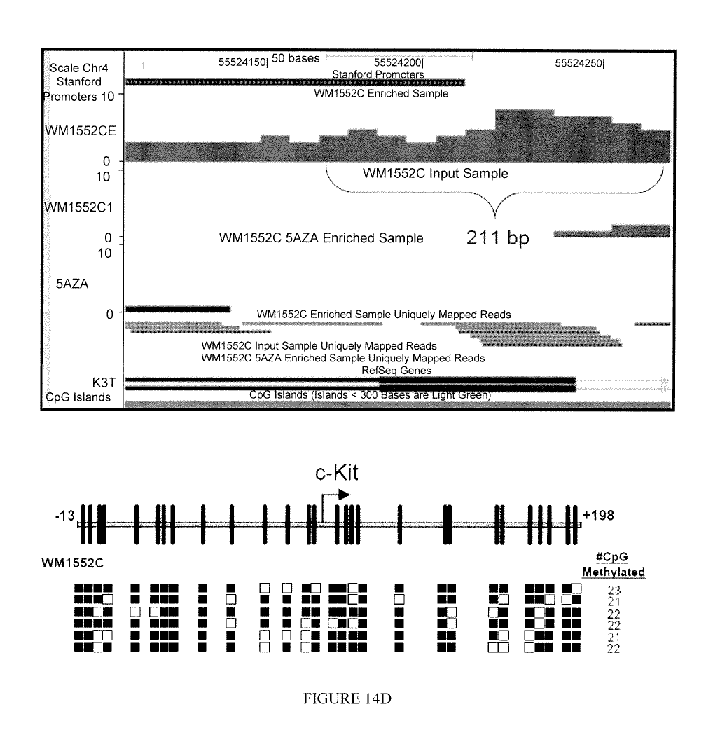

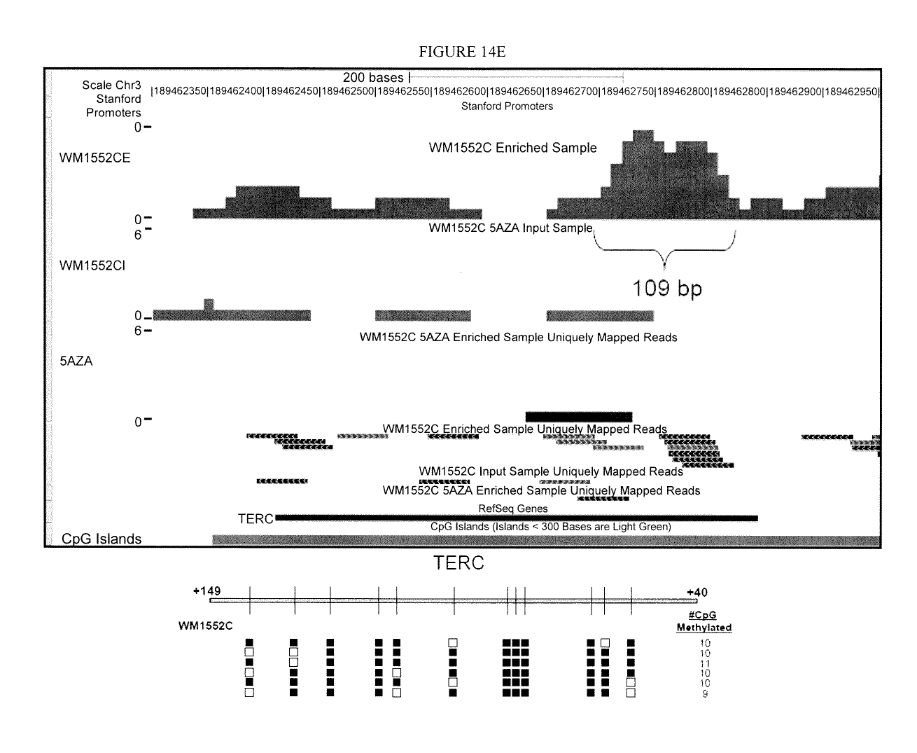

Gene regulation of telomerase RNA component (“TERC”) has also been identified herein as a being methylation-dependent. The non-coding RNA found on chromosome 3 (169482397-169482848) encoding TERC, a component of telomerase used to extend telomeres, was shown to be regulated by an upstream CpG island (see FIG. 14E). CHIP_Seq correlates methylation in an approximately 130 bp region near the transcriptional start site of this single exon ncRNA. The cell line WM1552C showed considerable downregulation of TERC, and up-regulation in Aza-treated WM1552C cells.

c-Kit

Proto-oncogene c-Kit is a cytokine receptor expressed on the surface of hematopoietic stem cells as well as other cell types. Altered forms of this receptor are known to be associated with some types of cancer. C-Kit is a receptor tyrosine kinase III, which binds to stem cell factor. It is located on chromosome 4 (55523022-55523948), and its regulation has been identified herein as being dependent upon methylation of an upstream CpG island (see FIG. 14D).

### QPCT

Glutaminyl-peptide cyclotransferase (“QPCT”) is a gene encoding human pituitary glutaminyl cyclase, which is responsible for the presence of pyroglutamyl residues in many neuroendocrine peptides. It is located on chromosome 2 (37571597-37571868), and its regulation has been identified herein as being methylation-dependent (see FIG. 14C).

### CYP1B1

Cytochrome P450, family 1, polypeptide B, family 1, (CYP1B1) is implicated in oxidation-reduction processes and electron carrier activity. Regulation of CYP1B1 has been shown herein to be dependent upon methylation of an upstream CpG island in its promoter region (see FIG. 14B).

### PCSK1

Proprotein convertase 1 (“PCSK1”) is an enzyme that performs the proteolytic cleavage of prohormones to their intermediate or completely cleaved forms. It is located on chromosome 5 (95768686-95769179), and regulation of the gene has been shown herein to be dependent upon methylation of an upstream CpG island in its promoter region (see FIG. 14A).

RNA Interference and siNA

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). Post-transcriptional gene silencing is believed to be an evolutionarily-conserved cellular mechanism for preventing expression of foreign genes that may be introduced into the host cell (Fire et al., 1999, Trends Genet., 15, 358). Post-transcriptional gene silencing may be an evolutionary response to the production of double-stranded RNAs (dsRNAs) resulting from viral infection or from the random integration of transposable elements (transposons) into a host genome. The presence of dsRNA in cells triggers the RNAi response that appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of dicer, a ribonuclease III enzyme (Bass, 2000, Cell, 101, 235; Zamore et al., 2000, Cell, 101, 25-33; Hammond et al., 2000, Nature, 404, 293). Dicer processes long dsRNA into double-stranded short interfering RNAs (siRNAs) which are typically about 21 to about 23 nucleotides in length and include about 19 base pair duplexes (Zamore et al., 2000, Cell, 101, 25-33; Bass, 2000, Cell, 101, 235; Elbashir et al., 2001, Genes Dev., 15, 188).

Single-stranded RNA, including the sense strand of siRNA, trigger an RNAi response mediated by an endonuclease complex known as an RNA-induced silencing complex (RISC). RISC mediates cleavage of this single-stranded RNA in the middle of the siRNA duplex region (i.e., the region complementary to the antisense strand of the siRNA duplex) (Elbashir et al., 2001, Genes Dev., 15, 188).

In certain embodiments, the siNAs may be a substrate for the cytoplasmic Dicer enzyme (i.e., a “Dicer substrate”) which is characterized as a double stranded nucleic acid capable of being processed in vivo by Dicer to produce an active nucleic acid molecules. The activity of Dicer and requirements for Dicer substrates are described, for example, U.S. 2005/0244858. Briefly, it has been found that dsRNA, having about 25 to about 30 nucleotides, effective result in a down-regulation of gene expression. Without wishing to be bound by any theory, it is believed that Dicer cleaves the longer double stranded nucleic acid into shorter segments and facilitates the incorporation of the single-stranded cleavage products into the RNA-induced silencing complex (RISC complex). The active RISC complex, containing a single-stranded nucleic acid cleaves the cytoplasmic RNA having complementary sequences.

It is believed that Dicer substrates must conform to certain general requirements in order to be processed by Dicer. The Dicer substrates must of a sufficient length (about 25 to about 30 nucleotides) to produce an active nucleic acid molecule and may further include one or more of the following properties: (i) the dsRNA is asymmetric, e.g., has a 3′ overhang on the first strand (antisense strand) and (ii) the dsRNA has a modified 3′ end on the antisense strand (sense strand) to direct orientation of Dicer binding and processing of the dsRNA to an active siRNA. The Dicer substrates may be symmetric or asymmetric. For example, Dicer substrates may have a sense strand includes 22-28 nucleotides and the antisense strand may include 24-30 nucleotides, resulting in duplex regions of about 25 to about 30 nucleotides, optionally having 3′-overhangs of 1-3 nucleotides.

Dicer substrates may have any modifications to the nucleotide base, sugar or phosphate backbone as known in the art and/or as described herein for other nucleic acid molecules (such as siNA molecules).

The RNAi pathway may be induced in mammalian and other cells by the introduction of synthetic siRNAs that are 21 nucleotides in length (Elbashir et al., 2001, Nature, 411, 494 and Tuschl et al., WO 01/75164; incorporated by reference in their entirety). Other examples of the requirements necessary to induce the down-regulation of gene expression by RNAi are described in Zamore et al., 2000, Cell, 101, 25-33; Bass, 2001, Nature, 411, 428-429; Kreutzer et al., WO 00/44895; Zernicka-Goetz et al., WO 01/36646; Fire, WO 99/32619; Plaetinck et al., WO 00/01846; Mello and Fire, WO 01/29058; Deschamps-Depaillette, WO 99/07409; and Li et al., WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831; each of which is hereby incorporated by reference in its entirety.

Briefly, an siNA nucleic acid molecule can be assembled from two separate polynucleotide strands (a sense strand and an antisense strand) that are at least partially complementary and capable of forming stable duplexes. The length of the duplex region may vary from about 15 to about 49 nucleotides (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides). Typically, the antisense strand includes nucleotide sequence that is complementary to nucleotide sequence in a target nucleic acid molecule. The sense strand includes nucleotide sequence corresponding to the target nucleic acid sequence which is therefore at least substantially complementary to the antisense stand. Optionally, an siNA is “RISC length” and/or may be a substrate for the Dicer enzyme. Optionally, an siNA nucleic acid molecule may be assembled from a single polynucleotide, where the sense and antisense regions of the nucleic acid molecules are linked such that the antisense region and sense region fold to form a duplex region (i.e., forming a hairpin structure).

### 5′ Ends, 3′ Ends and Overhangs

siNAs may be blunt-ended on both sides, have overhangs on both sides or a combination of blunt and overhang ends. Overhangs may occur on either the 5′- or 3′-end of the sense or antisense strand. Overhangs typically consist of 1-8 nucleotides (e.g., 1, 2, 3, 4, 5, 6, 7, or 8 nucleotides each) and are not necessarily the same length on the 5′- and 3′-end of the siNA duplex. The nucleotide(s) forming the overhang need not be of the same character as those of the duplex region and may include deoxyribonucleotide(s), ribonucleotide(s), natural and non-natural nucleobases or any nucleotide modified in the sugar, base or phosphate group such as disclosed herein.

The 5′- and/or 3′-end of one or both strands of the nucleic acid may include a free hydroxyl group or may contain a chemical modification to improve stability. Examples of end modifications (e.g., terminal caps) include, but are not limited to, abasic, deoxy abasic, inverted (deoxy) abasic, glyceryl, dinucleotide, acyclic nucleotide, amino, fluoro, chloro, bromo, CN, CF, methoxy, imidazole, carboxylate, thioate, C1 to C10 lower alkyl, substituted lower alkyl, alkaryl or aralkyl, OCF3, OCN, O-, S-, or N-alkyl; O-, S-, or N-alkenyl; SOCH3; SO2CH3; ONO2; NO2, N3; heterocycloalkyl; heterocycloalkaryl; aminoalkylamino; polyalkylamino or substituted silyl, as, among others, described in European patents EP 586,520 and EP 618,925.

### Chemical Modifications

siNA molecules optionally may contain one or more chemical modifications to one or more nucleotides. There is no requirement that chemical modifications are of the same type or in the same location on each of the siNA strands. Thus, each of the sense and antisense strands of an siNA may contain a mixture of modified and unmodified nucleotides. Modifications may be made for any suitable purpose including, for example, to increase RNAi activity, increase the in vivo stability of the molecules (e.g., when present in the blood), and/or to increase bioavailability.

Suitable modifications include, for example, internucleotide or internucleoside linkages, dideoxyribonucleotides, 2′-sugar modification including amino, fluoro, methoxy, alkoxy and alkyl modifications; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dide-oxythymidine (d4T).

Other suitable modifications include, for example, locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides (WO 00/47599, WO 99/14226, WO 98/39352, and WO 2004/083430).

Chemical modifications also include terminal modifications on the 5′ and/or 3′ part of the oligonucleotides and are also known as capping moieties. Such terminal modifications are selected from a nucleotide, a modified nucleotide, a lipid, a peptide, and a sugar.

Chemical modifications also include L-nucleotides. Optionally, the L-nucleotides may further include at least one sugar or base modification and/or a backbone modification as described herein.

Provided herein are methods for treating a patient diagnosed as having melanoma by administering to the patient an effective amount of a therapeutic agent that increases miR-34b expression. The therapeutic agent may increase miR-34b expression by, in further embodiments, at least 10%, at least 20%, at least 30%, at least 40%, at least 50%, at least 60% or at least 100%. Additionally, the therapeutic agent may decrease CpG island methylation, or may result in a reduction of miR-140 expression, at least 10%, at least 50%, or at least 90%. The therapeutic agent may be an anti-sense nucleic acid, which may, be encoded in a vector or a viral vector, or may be contained within a liposome.

### Delivery of Nucleic Acid-Containing Pharmaceutical Formulations

Nucleic acid molecules disclosed herein (including siNAs and Dicer substrates) may be administered with a carrier or diluent or with a delivery vehicle which facilitate entry to the cell. Suitable delivery vehicles include, for example, viral vectors, viral particles, liposome formulations, and lipofectin.

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995), Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000); U.S. Pat. Nos. 6,395,713; 6,235,310; 5,225,182; 5,169,383; 5,167,616; 4,959,217; 4,925,678; 4,487,603; and 4,486,194; WO 94/02595; WO 00/03683; WO 02/08754; and U.S. 2003/077829.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); WO 03/47518; and WO 03/46185), poly(lactic-co-glycolic)acid (PLGA) and PLCA microspheres (see for example U.S. Pat. No. 6,447,796 and U.S. 2002/130430), biodegradable nanocapsules, and bioadhesive microspheres, or by proteinaceous vectors (WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5: 2330-2337 (1999) and WO 99/31262. The molecules of the instant invention can be used as pharmaceutical agents.

Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly (ethylene glycol) lipids (PEG-modified, or long-circulating liposomes or stealth liposomes).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see, for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999., PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; U.S. Pat. No. 6,586,524 and U.S. 2003/0077829).

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Therapeutic nucleic acid molecules may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors are capable of expressing the nucleic acid molecules either permanently or transiently in target cells. Delivery of nucleic acid molecule expressing vectors can be systemic, such as by intravenous, subcutaneous, or intramuscular administration.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine. An expression vector may encode one or both strands of a nucleic acid duplex, or a single self-complementary strand that self hybridizes into a nucleic acid duplex. The nucleic acid sequences encoding nucleic acid molecules can be operably linked to a transcriptional regulatory element that results expression of the nucleic acid molecule in the target cell. Transcriptional regulatory elements may include one or more transcription initiation regions (e.g., eukaryotic pol I, II or III initiation region) and/or transcription termination regions (e.g., eukaryotic pol I, II or III termination region). The vector can optionally include an open reading frame (ORF) for a protein operably linked on the 5′ side or the 3′-side of the sequence encoding the nucleic acid molecule; and/or an intron (intervening sequences).

The nucleic acid molecules or the vector construct can be introduced into the cell using suitable formulations. One preferable formulation is with a lipid formulation such as in Lipofectamine™ 2000 (Invitrogen, CA, USA), vitamin A coupled liposomes (Sato et al. Nat Biotechnol 2008; 26:431-442, PCT Patent Publication No. WO 2006/068232). Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA in a buffer or saline solution and directly inject the formulated dsRNA into cells, as in studies with oocytes. The direct injection of dsRNA duplexes may also be done. Suitable methods of introducing dsRNA are provided, for example, in U.S. 2004/0203145 and U.S. 20070265220.

Polymeric nanocapsules or microcapsules facilitate transport and release of the encapsulated or bound dsRNA into the cell. They include polymeric and monomeric materials, especially including polybutylcyanoacrylate. The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid moles may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system. Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

### EXAMPLES

The present methods, thus generally described, will be understood more readily by reference to the following examples, which are provided by way of illustration and are not intended to be limiting of the present methods and kits.

### Example 1

### Identification of Epigenetically Regulated miRNAs in Melanoma Cell Lines

Human epidermal melanocyte cell line HEM-1 (ScienCell, Catalog #2200, grown in MelM media containing MelGS growth supplements, 0.5% fetal bovine serum (FBS), and penn/strep solution), human epidermal keratinocytes (HEK, ScienCell, Catalog #2100, grown in Keratinocyte Medium, ScienCell, Catalog #2101), and the melanoma cell lines WM793B (stage 1, Wistar Institute), WM278 (stage 2, Wistar Institute), WM1552C (stage 3, American Type Culture Collection Number: CRL-2808), and A375 (stage 4, American Type Culture Collection) were used in the present experiments. Melanoma cells were grown in Complete Tu Medium containing a 4:1 mixture of MCDB-153 medium with 1.5 g/L sodium bicarbonate and Leibovitz's L-15 medium with 2 mM L-glutamine, 2% FBS, and 1.68 mM CaCl₂. All clinical samples were graciously donated by Dr. James Goydos, Robert Wood Johnson Medical School.

Genomic DNA from cell lines was acquired from 10⁷ cells each, harvested by trypsinization, washed once in phosphate-buffered saline (PBS), and purified using the QiaAmp DNA mini kit (QIAGEN). DNA from 25 mg of patient samples was isolated by overnight incubation with proteinase K at 55° C., with subsequent purification using the QiaAmp DNA mini kit (QIAGEN). All samples were quantified using the ND-1000 spectrophotometer (Nanodrop). DNA (0.5 μg) was treated with sodium bisulfite using the EZ DNA methylation kit (Zymo Research) and eluted in 10 μL elution buffer.

WM1552C cells (5×10⁵) were plated into 75-cm² flasks. Each flask was treated with either 10 μg/mL 5-Aza-dC, 1 mM 4-PBA, or both. Each day for 5 days, the cells were washed with PBS, fed fresh media, and treated as above. After day 5, the cells were trypsinized, washed once with PBS, and spun down at 1200 rpm for 5 min. Cell pellets were prepared for total RNA using Trizol protocol (Invitrogen/Life Technologies), and RNA was quantified using the ND-1000 spectrophotometer (NanoDrop). The assay was performed in duplicate.

For cDNA synthesis and real-time PCR by Taqman Low Density Array (TLDA), total RNA (800 ng) was subjected to 8 separate reverse transcription reactions (100 ng each) using Multiplex RT for TaqMan® MicroRNA Assays, Human Pool Set kit (Applied Biosystems/Life Technologies). The resulting cDNA (10 μL) was diluted 1:62.5 with nuclease-free water. TLDAs consisting of a panel of 365 human miRNAs and 3 miRNA endogenous controls were run in triplicate for each sample. Diluted cDNA (50 μL) was added to 50 μL of 2× TaqMan Universal Master Mix (No AmpErase UNG; Applied Biosystems/Life Technologies). This 100-4, mixture was applied to the respective array port and the TLDA was then centrifuged twice and sealed. Quantitative real-time PCR was performed using the Applied Biosystems 7900 Real-Time PCR Sequence Detection System with the following thermal cycling parameters: 94.5° C. for 30 s followed by 40 cycles of 97° C. for 30 s, 59.7° C. for 1 min.

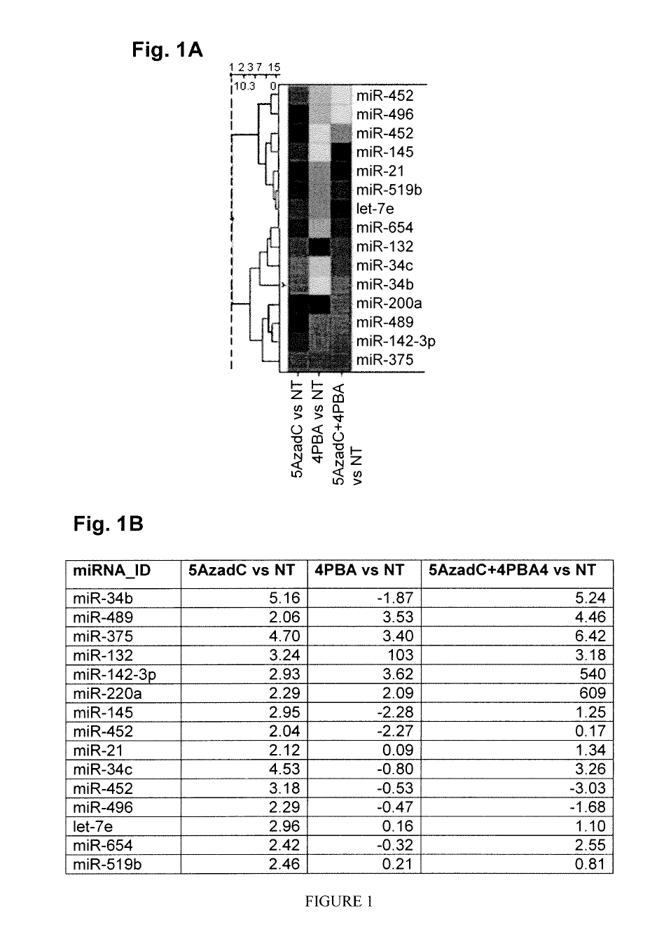

To identify transcriptional changes in melanoma cells induced by DNA methylation and/or histone modification, WM1552 cells were treated as above with 5AzadC, 4-PBA, or with both. Following exposure, cellular miRNA expression was compared between treated and untreated cells using two miRNA expression profiling platforms (TILDA; ABI and NCode; Lifetech). Both platforms identified the same miRNAs as being upregulated in WM1552C cells after treatment with 5AzadC and/or 4-PBA. FIG. 1(A) shows differential miRNA expression represented as hierarchical clustering and (B) shows differential miRNA expression after confirmatory quantification by qRT-PCR. Notable increases in expression were observed for miR-34b, miR-375, miR-132, miR-200a, miR-145, miR-452, miR-21, miR-34c, miR-496, miR-let7e, miR-654, miR-519b, miR-142-3p, and miR-489, in particular (see Table 1). Several of the upregulated miRNAs have previously been shown by bioinformatic analysis to contain CpG islands in their putative regulatory regions (Saini et al., 2007), which indicates that reactivation of miRNA expression in WM1552C cells may be epigenetically controlled.

TABLE 1

| | | |
|---|---|---|
| SEQ ID NO: 2 | miR-34b | CAAUCACUAACUCCACUGCCAU |
| SEQ ID NO: 4 | Mir-let-7e | UGAGGUAGGAGGUUGUAUAGUU |
| SEQ ID NO: 5 | Mir-21 | UAGCUUAUCAGACUGAUGUUGA |
| SEQ ID NO: 1 | miR-375 | UUUGUUCGUUCGGCUCGCGUGA |

### Example 2

### CpG Island Methylation in Melanocytes, Keratinocytes, and Melanoma Cells

Bisulfite-treated genomic eluate (2 μL) was used for bisulfite PCR using the following primers: miR-375 For (GGT GGC TGG GAA AGG AGG GG; SEQ ID NO:6) and miR-375 Rev (GGC TGG TGC TGA GAG GCC GCC CCT GCC TCA; SEQ ID NO:7) to produce a 278-bp product. PCR was performed using a 6-minute hot-start at 95° C., followed by 35 cycles at 94° C. for 20 s, 54° C. for 25 s, and 72° C. for 30 s, ending with a 10-min extension at 72° C. using AmpliTaq Gold (Applied Biosystems/Life Technologies). PCR products were gel purified using the QiaQuick gel extraction kit (QIAGEN) and cloned into the pCR4-TOPO vector (Invitrogen/Life Technologies). Nine clones for each cell line and 6 clones for each patient sample were sequenced using M13 primers and the BigDye terminator kit v1.1 (Applied Biosystems/Life Technologies), analyzed on a 3130x1 Genetic Analyzer (Applied Biosystems/Life Technologies), and aligned using Vector NTi AlignX (Invitrogen/Life Technologies).

Northern blot analysis was performed using 20 μg of total RNA concentrated from each sample (untreated, 5AzadC, PBA, or both treatments on melanoma cells as described above). Samples were separated on a 15% TBE-urea polyacrylamide gel by electrophoresis and electroblotted to nylon membranes, followed by cross-linking under UV light. The membranes were then prehybridized in Ultrahyb-Oligo (Ambion) for 30′ at 42° C. and hybridized with 5′-biotinylated anti-miRNA DNA oligonucleotides (100 nM each) at 42° C. overnight. Analysis of the membranes was performed by washing and chemiluminescence detection using the Brightstar Detection kit (Ambion). An anti-U6 probe was used as a reference control (at 10 pM).

Epigenetically regulated miRNAs often carry modifications such as cytosine methylation in CpG islands located in their upstream regulatory regions. To determine if this was the case for miR-375, methylation patterns within the CpG islands located 1.0 Kb upstream of miRNA-375 in melanoma cells were examined, along with normal melanocytes and keratinocytes for controls. Genomic DNA from the cell lines above were bisulfite treated, cloned, and sequenced. The sequence data was then compared to that of untreated genomic DNA.

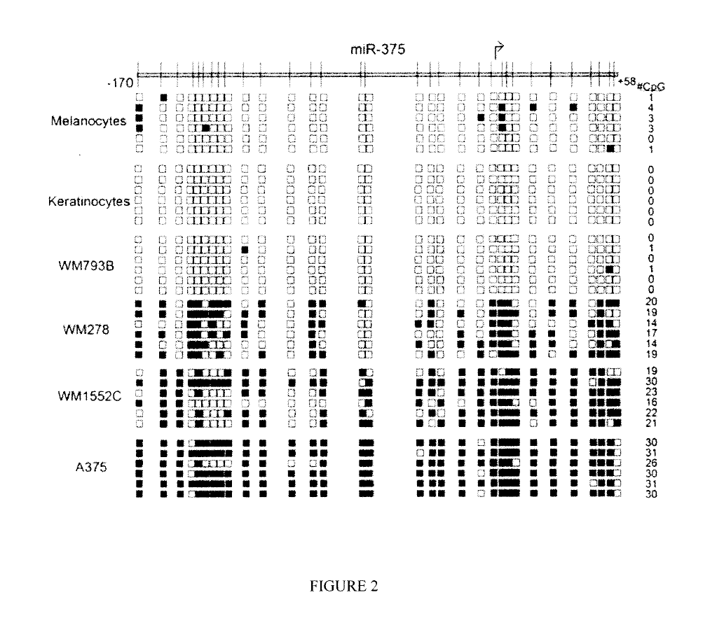

Several CpG islands were identified in the upstream region of miR-375, one of which is located from −170 to +58 bp upstream of miR-375 and contains 32 CpG dinucleotides, as shown in FIG. 2, and formed the basis of the present assay. As shown in FIG. 2, the CpG island was entirely unmethylated in keratinocytes (0.0%), and only poorly methylated in the stage I melanoma cell line WM793B (0.5%) and in melanocytes (6.2%). In contrast, extensive methylation was detected in DNA from the stage II melanoma line WM278 (57.3%), the stage III line WM1552C (68.2%), and the stage IV line A375 (92.7%). These results indicate that the proportion of methylated CpG dinucleotides in the miR-375 island increased with advancing melanoma stage and that epigenetic changes in this CpG island may be stage specific.

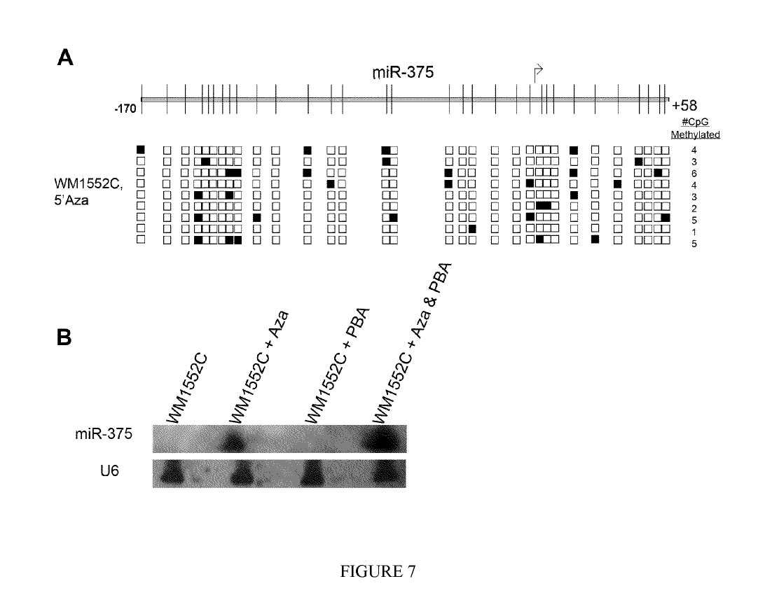

To confirm that methylation of CpG islands is responsible for downregulation of miR-375 expression in melanoma, the effect of 5AzadC treatment on the methylation state of the region was first confirmed. Genomic DNA from 5AzadC-treated WM1552C cells was treated with bisulfite and sequenced. FIG. 7(A) shows that 5AzadC treatment greatly reduced methylation levels from 68.2% to 16.7% at this location. As described above, WM1552C cells were treated with 5AzadC, 4-PBA or both, to determine their effect on miR-375 expression as measured by Northern blotting, the results of which are shown in FIG. 7(B).

The results indicate that miR-375 expression was induced by 5AzadC but not by 4-PBA treatment. However, a synergistic effect of 5AzadC and 4-PBA was observed, resulting in greater induction of miR-375 than after treatment with 5AzadC alone. These data show that demethylation of miR-375 induces its expression, confirming that miR-375 is epigenetically regulated.

### Example 3

### miR-375 CpG Island Methylation in Melanoma Patients and Normal Skin

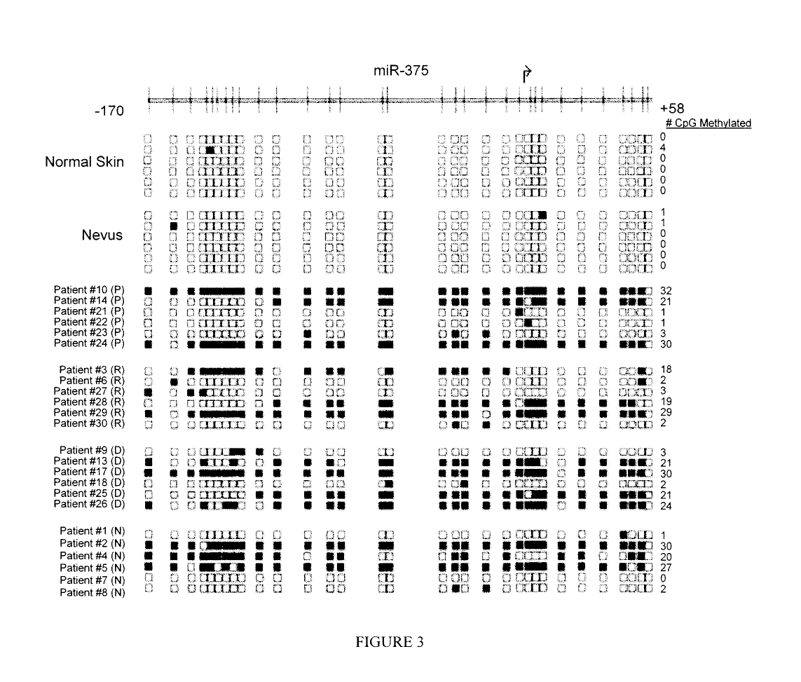

To confirm the pathological relevance of the finding that miR-375 is epigenetically regulated, CpG island methylation for miR-375 was then measured in ex-vivo tissue samples from melanoma patients and normal skin and nevi samples. In samples of normal skin and nevi the miR-375 CpG island was almost entirely unmethylated, as shown in FIG. 3, consistent with the lack of methylation of keratinocytes, as shown in FIG. 2.

Twenty-four genomic DNA patient samples from four groups of melanoma patients (primary melanoma, regional metastases, distant metastases, and nodal metastases, as shown in FIG. 3) were then analyzed for methylation detection for miR-375 by CpG methylation analysis using pyrosequencing (PyroMark MD, Biotage/QIAGen). 500 ng of genomic DNA was bisulfite treated using the EZ DNA Methylation Kit (Zymo Research), utilizing forward (5′-AGG GTG GTT GGG AAA GGA G-3′; SEQ ID NO:8) and reverse tailed primers (5′-CTA AAA AAC CGC CCC TAC CTC A-3′; SEQ ID NO:9) (EpigenDx). This produced an amplicon size of 272 bp. 10 μl of PCR product was prepared for pyrosequencing by binding to streptavidin-coated Sepharose beads, separating the dsDNA by NaOH denaturation, washing away the non-biotinylated DNA, and finally annealing the biotinylated ssDNA to the sequencing primer (0.5 μM). Three different pyrosequencing primers were used for methylation detection of miR-375 resulting in a total of 21 CpG sites analyzed. The sequencing primers used were as follows: FS2 primer (5′-GTG TTA GTY GTA GAT GAG TTT A-3′; SEQ ID NO:10), FS3 primer (5′-GAT TAG GAT TAG GAG ATT AT-3′; SEQ ID NO:11), and FS4 primer (5′-GAT GAG TTT TTT GTA TAA AT-3′; SEQ ID NO:12). High-resolution, high-throughput DNA methylation analysis was performed by using the Pyro Q-CpG Software (Biotage/QIAGEN).

The results indicated that three of the six primary melanoma tissues (P), three of the six regional metastases (R), four of the six distant metastases (D), and three of the six nodal metastases (N) were hypermethylated, with average hypermethylation of 77.4%. CpG methylation in these samples was confirmed by DNA pyrosequencing as described above and in Table 4. In addition to patient samples, DNA from normal skin, nevus, and melanoma cell lines WM793B (stage I) and A375 (stage IV) cell lines were pyrosequenced. Table 4 shows that the results correlated well with previous bisulfite sequencing results and revealed low methylation levels in normal skin and nevus, with average methylation of 2.6% and 3.9%, respectively. As expected, high methylation levels were detected in stage IV cells (86.8%). Of the patient samples, 12 of the 13 samples positively identified by bisulfite sequencing as having hypermethylated CpG were confirmed by pyrosequencing (with a positive match cut-off set at 20%, only regional metastatic patient sample #3 fell below with an average methylation at 19.33%). Collectively, these results show that the methylation state of miR-375 CpG islands increases consistently with the transition from normal skin to melanoma, and further indicates the diagnostic potential of this marker for pathological staging of melanoma.

TABLE 4
miR-375% Methylation

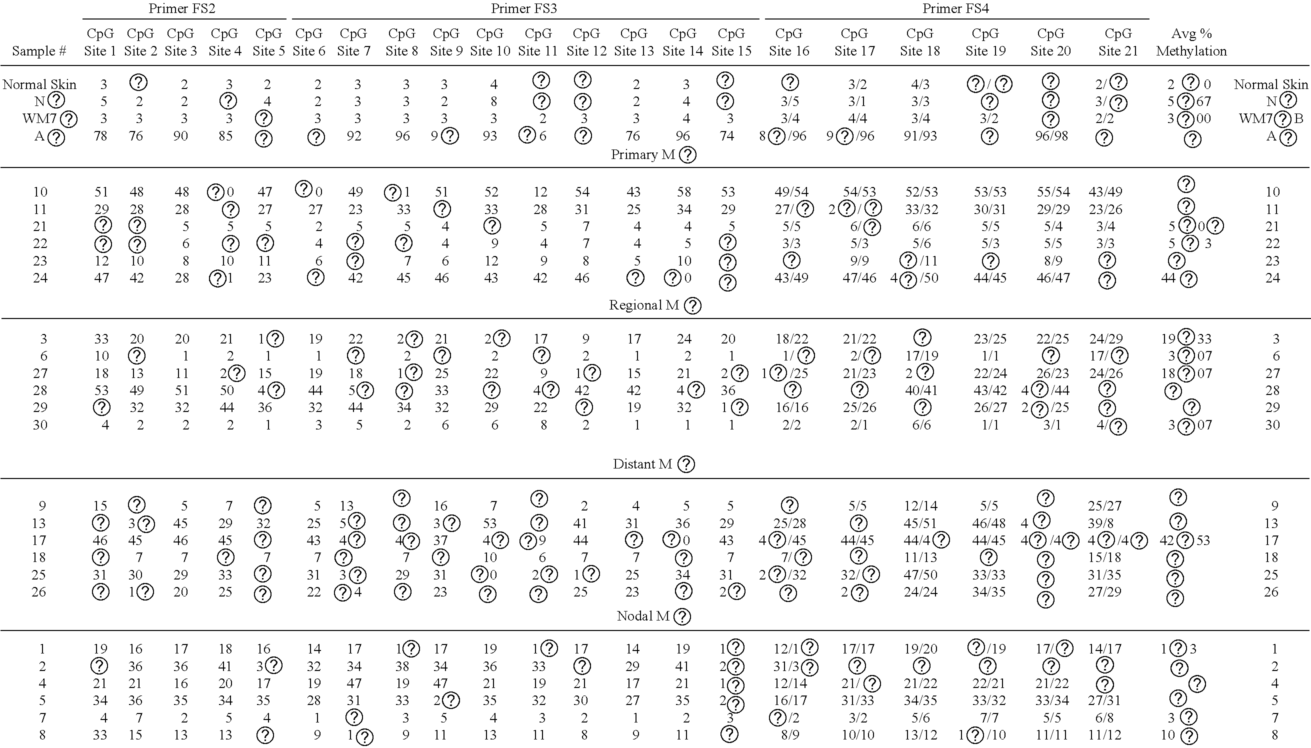

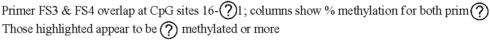

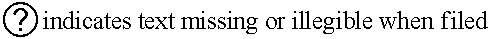

### Example 4

### CpG Islands Methylation at the 5′ Upstream Region of miR-34B in Melanoma

Bisulfite-treated genomic eluate (2 μL) was used for bisulfite PCR using the following primer combinations: let7i f1 (GGGGGTAGTT TAGAATTAGT TGGTGTTTG; SEQ ID NO:13) and let7i r1 (CCCCTTCTTT TCCTTTACCT TCCC; SEQ ID NO: 14) to produce a 301-bp product, 124a No-C-For (GGAAAGGGGA GAAGTGTGGG; SEQ ID NO: 15) and 124-3 Rev (CACCGCGTAC CTTAATTATA TAAAC; SEQ ID NO: 16) to produce a 260-bp product, & miR34b f1 (GAATTTGGGT TTTTATTTTT TAGG; SEQ ID NO: 17) and miR34b r1 (CCAAACCCTA AAACTAACTC TCTC; SEQ ID NO: 18) to produce a 236-bp product. PCR was performed using a 6-minute hot-start at 95° C., followed by 35 cycles at 94° C. for 20 s, 54° C. for 25 s, and 72° C. for 30 s, ending with a 10-min extension at 72° C. using AmpliTaq Gold (Applied Biosystems/Life Technologies). PCR products were gel purified using the QiaQuick gel extraction kit (QIAGEN) and cloned into the pCR4-TOPO vector (Invitrogen/Life Technologies). Nine clones for each miRNA candidate and 6 clones for each patient sample were sequenced using M13 primers and the BigDye terminator kit v1.1 (Applied Biosystems/Life Technologies), analyzed on a 3130×1 Genetic Analyzer (Applied Biosystems/Life Technologies), and aligned using Vector NTi AlignX (Invitrogen/Life Technologies).

Total RNA concentrated from each sample (20 ng from cell lines or 5-Aza-dC-treated melanoma cells as above), was analyzed by northern blot and separated in 15% TBE-urea polyacrylamide gels by electrophoresis. The RNA was electroblotted onto nylon membranes, cross-linked by ultraviolet light, prehybridized in Ultrahyb-Oligo (Ambion) for 30 min at 42° C., and hybridized with 5′-biotinylated anti-miRNA DNA oligonucleotides (100 nM each) at 42° C. overnight. The blots were then washed, and the signal was detected by chemiluminiscence (Brightstar Detection kit, Ambion). Anti-U6 probes (10 pM) were used as a reference control.

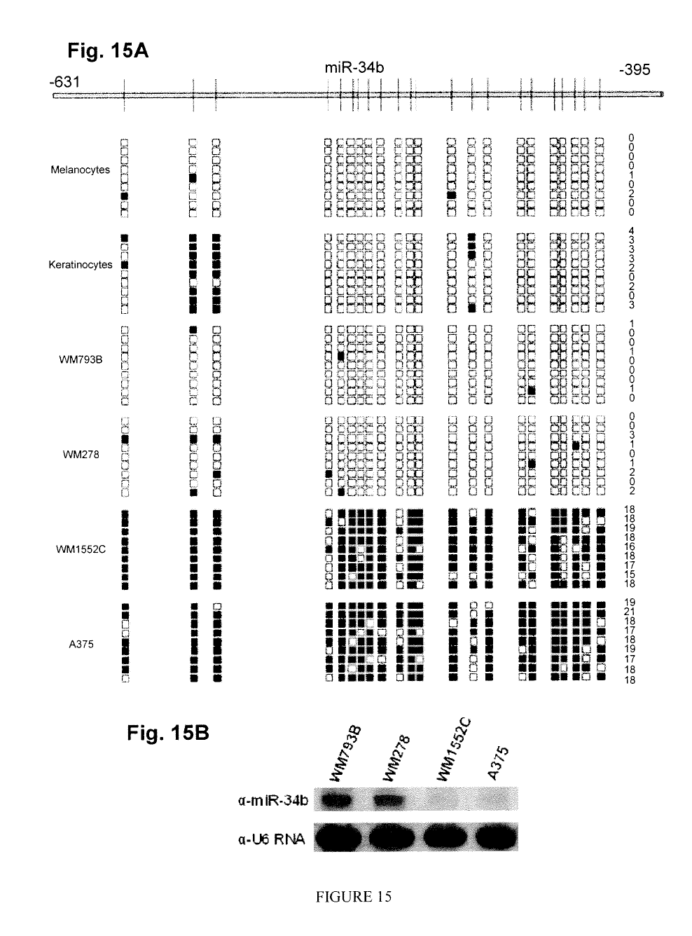

miRNAs that are modulated by 5-Aza-dC are likely to carry epigenetic modifications in the CpG islands located in their regulatory elements. To identify the CpG island methylation in miR-34b, 2 kb of the 5′ upstream sequences (5′-UPS) known to contain CpG islands were examined. miR-34b, whose expression was strongly modulated by 5-Aza-dC (FIG. 1B), contains several CpG islands in its 5′ UPS, including a distinct CpG island located between −631 and −395 bp that contains 22 CpG dinucleotides. To examine the methylation patterns within the CpG islands, the genomic DNA of both melanocytes and WM1552C cells were sequenced after bisulfite treatment. The sequence data were then compared to that of untreated genomic DNA isolated from the same cells. Though a negligible CpG island methylation was observed in normal melanocyte and keratinocyte cell lines, or in melanoma cell lines obtained from stage 1 (WM793B) and the stage 2 (WM278) melanoma tumors, this region was found to be highly methylated in cell lines obtained from stage 3 (WM1552C) and stage 4 (A375) melanoma tumors (FIG. 15A). Further examination by RNA gel blot analysis (FIG. 15B) revealed that miR-34b expression correlated inversely with the level of its methylation: stage 1 and 2 melanoma cells showed high miR-34b expression while stage 3 and 4 had significantly lower levels of expression.

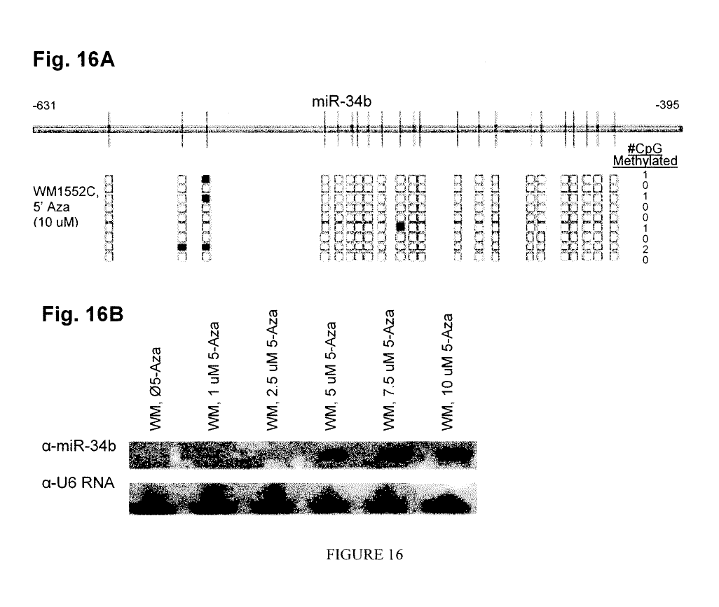

Upon treatment of WM1552C with increasing concentrations of 5-Aza-dC, expression of miR-34b was induced, and the maximum induction was observed at 10 μM of 5-Aza-dC (FIG. 16B). To determine whether the induction of miR-34b expression by 5-Aza-dC is associated with demethylation at the CpG island in the 5′ UTR of miR-34b, the miR-34b genomic region of treated cells was sequenced by bisulfite sequencing. CpG island methylation was largely erased by the 5-Aza-dC treatment (FIG. 16A). These results support the view that miR-34b is epigenetically regulated in human melanomas. Next, methylated DNA regions were isolated using MethylMiner™ kit (Life Technologies) with single high-salt (2 M NaCl) elution step and subjected DNA to next-generation DNA sequencing to identify highly methylated regions in the melanoma genome. FIG. 1C shows the methylation of miR-34b and -34c in the 5′ upstream genomics sequences.

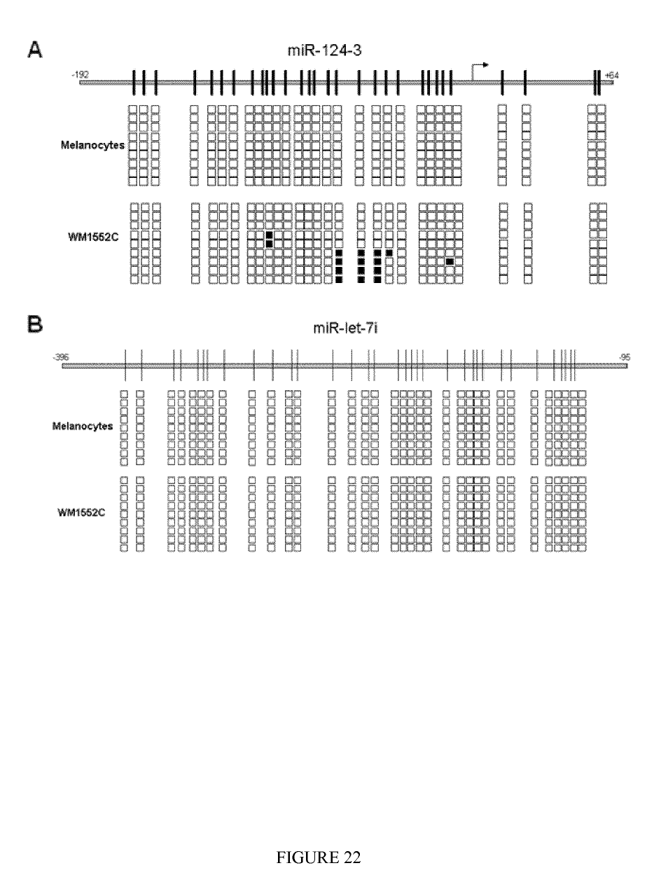

Using the above techniques, it was determined that miRNAs that contain CpG islands in their 5′ upstream region are not always methylated in melanomas. For an example, the expression and methylation levels of two miRNA genes (miR-124-3a and miR-let-7i) that are known to contain CpG islands in their 5′ UPS (Zhou et al., 2007) were not affected by the 5-Aza-dC treatment. Bisulfite sequencing further confirmed that these CpG islands were not detectably methylated in melanoma cell lines under standard growth conditions (FIG. 22).

### Example 5

### miR-34b CpG Island Methylation in Melanoma Patients and Normal Skin

Genomic DNA (melanocytes, WM1552C and WM1552C+5-Aza-dC) was fragmented to 50-400 bp (mean ˜250 bp) using a Covaris™ S2 System (Woburn, Mass.), and 10 μg was subjected to MBD-protein capture with the MethylMiner™ Methylated DNA Enrichment Kit (Life Technologies) following the recommended protocol. The methylated DNA was resuspended in 40 μL GibcoR UltraPure™ DNase/RNase-Free Distilled Water (Life Technologies) and quantified by UV absorbance spectroscopy. For single-fraction elution, buffer containing 2 M NaCl was used to elute methylated DNA captured using the MethylMiner™ kit. The methylated DNA fragments recovered were ethanol precipitated and resuspended. This DNA, and a sample of DNA that did not undergo enrichment with the MethylMiner™ kit (whole-genome, unenriched), were used for SOLiD™ System fragment library construction, which includes a gel-based size selection step to obtain a mean insert length of ˜150 bp.

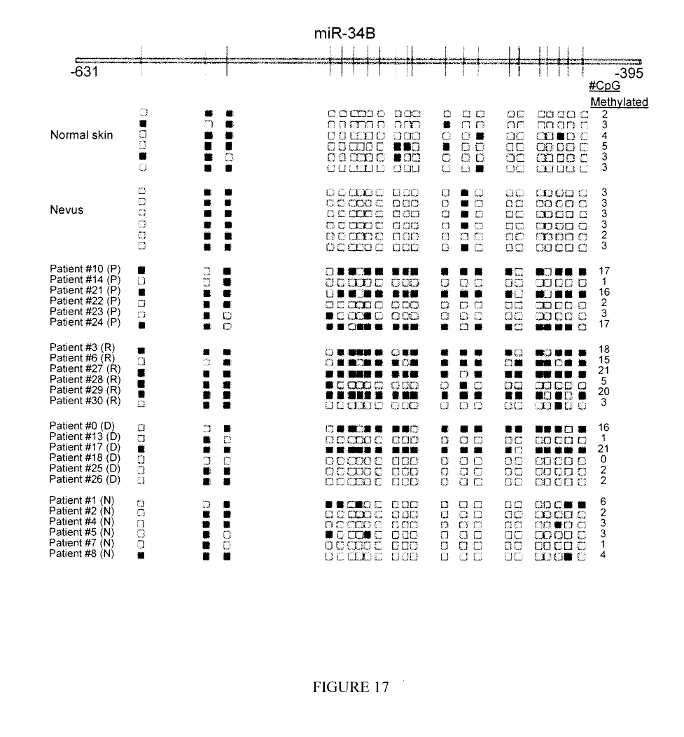

Bisulphite conversion and sequencing of normal skin samples and nevi revealed that the 5′ upstream sequences of miR-34b were largely hypomethylated (FIG. 17), except at two sites located near the 5′ end (sites #2 and 3). Hypermethylation at these two sites was also observed in keratinocytes (FIG. 15A), suggesting that CpG methylation at these two sites may not be melanoma-specific, and therefore, may not correlate with oncogenic transformation.

CpG island methylation of miR-34b was examined in 24 melanoma patient samples separated into four groups: (a) primary melanoma, (b) regional metastases, (c) distant metastases, and (d) nodal metastases. Three out of six primary, four of six regional metastases, and two of six distant metastases melanoma samples were associated with hypermethylated CpG islands. Interestingly, none of the nodal metastases showed any significant degree of methylation in this region.

### Example 6

### Artificial Expression of miR-34B in Melanoma Cells Modulates Coding and Non-Coding RNA Genes

Total RNA was isolated by the Trizol method (Invitrogen/Life Technologies) with subsequent quantification and integrity analysis performed using an Agilent 2100 Bioanalyzer (Agilent Technologies, Santa Clara, Calif., USA). Total RNA (100 ng) was reverse transcribed using a High Capacity cDNA kit (Applied Biosystems/Life Technologies), and quantitative reverse-transcription PCR was carried out using TaqMan miRNA or mRNA Assays or SYBR Green mRNA Assays and a 7500 Real-Time PCR System (Applied Biosytems/Life Technologies) in accordance with the manufacturer's protocols. SYBR Green primers include CDC42 (CDC42 qPCR For-CTGCACCTAC CCACATGCAC TCGT [SEQ ID NO:19] and CDC42 qPCR Rev-TTAACTAGTA CTGGGAGGGG GAAGGG [SEQ ID NO:20]), FN1 (FN1 qPCR For-GGCTGACAGA GAAGATTCCC GAGAG [SEQ ID NO:21] and FN1 qPCR Rev-CCAGTTTAGA TGGATCTTGG CAGAGAGAC [SEQ ID NO:22]), and THBS2 (THBS2 qPCR For-TTACCGCTTC GTGCGCTTTG AC [SEQ ID NO:23] and THBS2 qPCR Rev-AACAGCGTGC CCCTGGACTT G [SEQ ID NO:24]). SDS1.2.3 software (Applied Biosystems/Life Technologies) was used for comparative Ct analysis, with RNU48, GAPDH, or β-actin serving as the endogenous controls.

Oligonucleotides complimentary to the hsa-miR-34b genomic sequences were constructed (miR-34b pre For-GTGCTCGGTT TGTAGGCAGT G [SEQ ID NO:25] and miR-34b pre Rev-GTGCCTTGTT TTGATGGCAG TG [SEQ ID NO:26]), containing HindIII and BamHI sites on their respective 5′ and 3′ ends, then amplified from melanocyte genomic DNA (Amplitaq Gold, Applied Biosystems/Life Technologies). The product was then TOPO cloned into pCR4-TOPO (Invitrogen/Life Technologies). The vector construct was sequenced and the pre-hsa-miR-34b fragment was sub-cloned into pcDNA4/H1-3TetR using the HindIII and BamHI sites to create pcDNA4/H1-3TetR/miR-34b. WM1552C melanoma cells (2.5×10⁵) were seeded into a single well of a 6-well plate and transfected with 5 μg pcDNA4/H1-3TetR (Vector Control) or pcDNA4/H1-3TetR/miR-34b using Fugene 6 (Roche). The following morning, cells were selected with 600 μg/mL Zeocin for the following 15 days. The remaining stable cells were then expanded and named WM1552C/34b and WM1552C/VO (vector only).

Total RNA was isolated from samples using Trizol (Invitrogen/Life Technologies) and fragmented through RNase III digestion. SOLiD sequence protocols (Applied Biosytems/Life Technologies) require reverse transcription of these RNAs, priming with a ligated primer, and the resulting cDNA amplified and size-selected in a 6% urea gel with the help of SYBR Gold dye for sequencing. The sequence libraries (150-200 bp size fragments) were further amplified using a bead-based emulsion PCR optimized to physically isolate a single bead/cDNA molecule. This enables massively parallel amplification of monoclonal DNA species. For RNA-seq experiments, we deposited approximately 90 million beads per sample onto a glass slide and analyzed them using a ligation-based sequencing technology. Data were mapped to the human genome using both BioScope and BFAST software packages and analyzed at the Burnham Institute Bioinformatics core facility.

Mapping of the SOLiD sequence data was performed using the standard pipeline of Blat-like Fast Accurate Search Tool (BFAST) (Horner et al., 2009) against the human genome (hg19) (Rhead et al., 2010). The BFAST algorithm first creates indices of the human genome using different masks (in this work, all 10 masks suggested in the BFAST manual were used). It then hashes the reads to a number of genomic locations based on the indices and performs detailed alignments between the reads and the genomic sequence at the hashed locations. The final output of BFAST contains the mapped genomic locations of each read and the qualities (represented via alignment scores) of the mappings.

The expression level of a gene can be calculated based on the mapping results. The BFAST algorithm successfully mapped ˜63% of the reads to the reference genome; among the mapped reads, ˜89% were uniquely mapped. Only uniquely mapped reads were considered to eliminate possible noise. For each gene (from the RefSeq annotation for hg19), a score was calculated as reads per kilobase of sequence per million reads (RPKM) (Mortazavi et al., 2008).

The predefined mapping protocol for SOLiD sequencing on BioScope 1.0 was also used. BioScope was able to map ˜63% of the reads to the reference genome, ˜83% uniquely. The output was then fed into the Partek Genomics Suite for analysis. Again, an RPKM score was computed for each gene.

The results of the two analysis pipelines were compared to arrive at a consensus. Fold-change was computed for each gene, this was a ratio of the WM1552C/34b RPKM over the WM1552C/VO RPKM. For this calculation, a pseudo-count of 1.0 was used to overcome divide by zero errors and to normalize the data at low RPKM values. A list of genes was produced such that both programs agreed on the regulation direction (i.e. both up-regulated or down-regulated). The order of the genes was then directed by the corroborated fold change. For the open reading frames, a cutoff was established with a minimum fold change of +/−1.5 (1.5 for miRNAs, −1.2 for target genes). The transcript delta was also obtained; this was used to select genes for further testing, this cutoff was set at 10.0 for open reading frames (1.3 for miRNAs, −2.0 for target genes). See Table 2. Of particular interest are the target genes of miR-34b. This target gene list was established from TargetScan 5.1 (Friedman et al., 2009) utilizing the top 500 miR-34b targets, irrespective of conservation. The data acquired from deep sequencing was then filtered through the target list to arrive at a final putative target list.

TABLE 2

| | |
|---|---|
| Bioscope | BFAST |

| | Fold | Transcript | Fold | Transcript |
|---|---|---|---|---|
| Gene Name | Change | Difference | Change | Difference |
| FCRLA | 2.85 | 17.76 | 2.84 | 14.95 |
| MMPS | 2.80 | 12.14 | 2.86 | 10.55 |
| S100B | 2.45 | 70.71 | 2.35 | 59.92 |
| RCANI | 2.38 | 44.75 | 2.34 | 43.98 |
| SERPINA3 | 2.37 | 13.31 | 2.45 | 10.54 |
| ACAN | 2.19 | 16.51 | 2.20 | 18.43 |
| HLA-DRBI | 2.18 | 22.90 | 2.27 | 15.71 |
| MIA | 1.90 | 37.10 | 1.90 | 31.30 |
| COL9A3 | 1.84 | 21.05 | 1.78 | 13.40 |
| IGFBP2 | 1.80 | 60.38 | 1.75 | 46.90 |
| CADM4 | 1.79 | 12.86 | 1.79 | 10.14 |
| FASN | 1.73 | 18.85 | 1.72 | 15.03 |
| HLA-DRA | 1.73 | 23.99 | 1.76 | 20.43 |
| RPL37A | 1.70 | 41.46 | 1.67 | 25.39 |
| MARCKSLI | 1.68 | 12.59 | 1.68 | 10.96 |
| KLF9 | 1.61 | 14.83 | 1.60 | 12.71 |
| NT5E | 1.55 | 18.87 | 1.53 | 15.25 |
| THBS2 | 1.55 | 123.34 | 1.53 | 100.99 |
| RPL24 | 1.52 | 16.61 | 1.79 | 12.58 |
| MMP14 | −1.54 | 14.10 | −1.54 | 11.92 |
| CAVI | −1.55 | 18.48 | −1.52 | 15.33 |
| TNS3 | −1.56 | 37.76 | −1.57 | 32.33 |
| CXCL1 | −1.57 | 164.16 | −1.55 | 136.23 |
| TGFBI | −1.58 | 23.76 | −1.60 | 19.23 |
| COL6A1 | −1.60 | 11.91 | −1.64 | 10.43 |
| SEMA3C | −1.61 | 14.88 | −1.61 | 12.41 |
| CYR61 | −1.63 | 31.05 | −1.60 | 25.31 |
| PDE1C | −1.66 | 16.34 | −1.67 | 13.00 |
| CRIMI | −1.66 | 13.32 | −1.69 | 11.42 |
| ITGB5 | −1.71 | 13.47 | −1.67 | 10.36 |
| IL6 | −1.72 | 15.64 | −1.62 | 11.43 |
| PTTGHP | −1.73 | 19.63 | −1.73 | 16.78 |
| FSTL1 | −1.74 | 25.38 | −1.76 | 21.59 |
| LOXL2 | −1.75 | 60.13 | −1.79 | 52.37 |
| UGCG | −1.76 | 19.21 | −1.76 | 14.74 |
| AP2M1 | −1.82 | 15.15 | −1.84 | 23.31 |
| IGFBP7 | −2.09 | 50.32 | −2.12 | 43.32 |
| WNT5A | −2.16 | 18.11 | −2.19 | 16.19 |
| ALCAM | −2.33 | 12.29 | −2.31 | 10.22 |
| TXNIP | −2.33 | 30.21 | −2.34 | 26.14 |
| FBNI | −2.55 | 25.18 | −2.54 | 20.45 |
| ID3 | −2.71 | 19.97 | −2.63 | 16.00 |
| S100A16 | −2.86 | 27.00 | −2.84 | 23.09 |
| ITGA3 | −3.63 | 18.17 | −3.42 | 21.36 |
| SERPINB2 | −3.68 | 14.12 | −3.64 | 14.77 |
| LYPD1 | −3.86 | 14.43 | −3.98 | 16.85 |
| TNFRSF21 | −4.00 | 25.16 | −3.92 | 22.18 |
| STC2 | −4.44 | 12.45 | −4.48 | 11.00 |
| SRGN | −5.87 | 17.37 | −5.67 | 14.98 |
| IL1B | −7.58 | 221.43 | −8.10 | 185.64 |
| DKK1 | −9.27 | 18.26 | −9.27 | 15.87 |
| INHBA | −9.48 | 12.28 | −8.84 | 9.96 |
| STC1 | −22.72 | 11.88 | −21.23 | 10.85 |

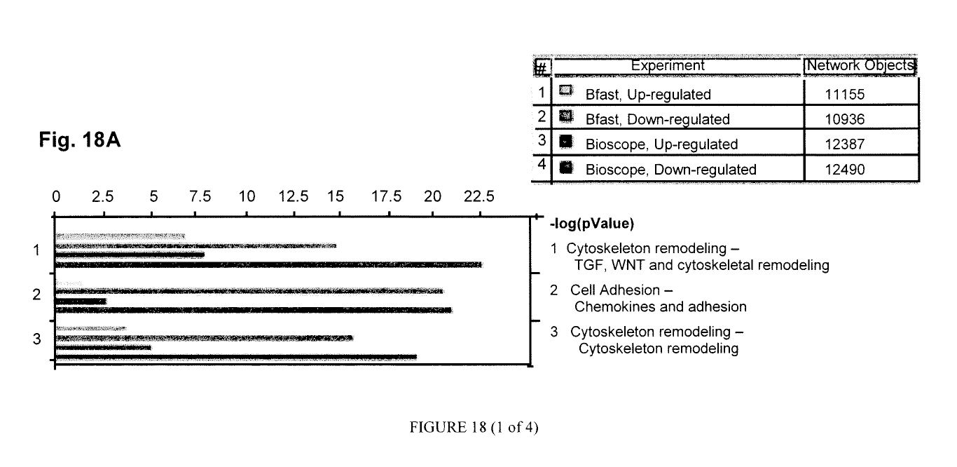

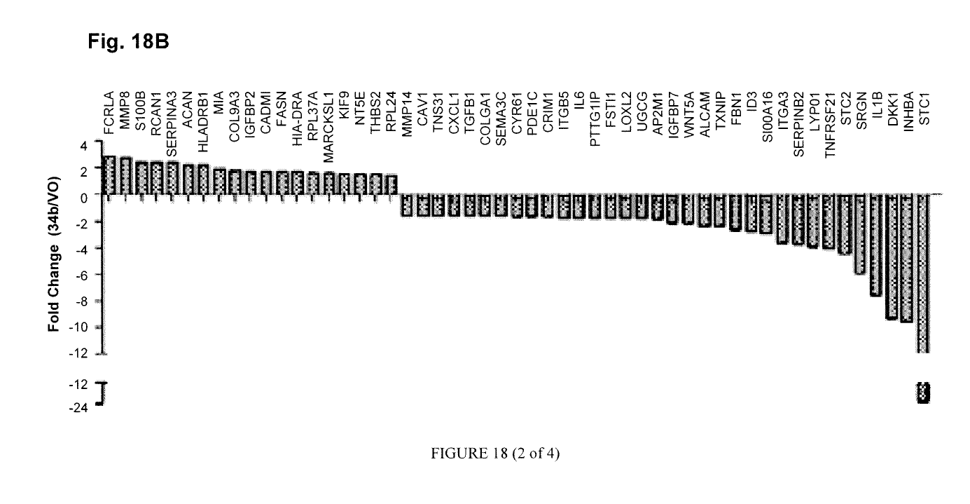

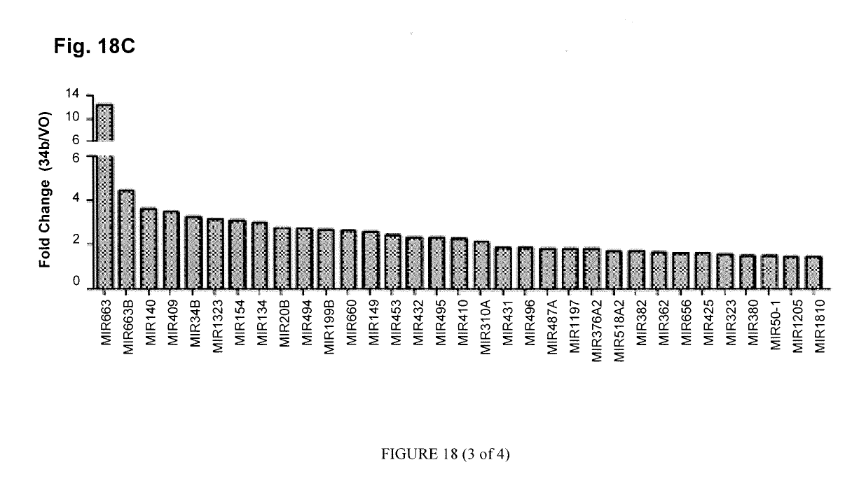

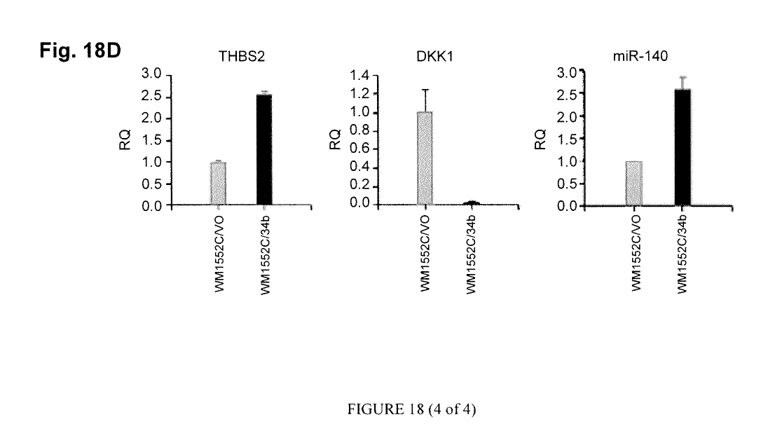

To understand the significance of miR-34b in malignant melanomas, miR-34b was stably expressed in the stage 3 melanoma cell line WM1552C (WM1552C/34b). Total RNA was isolated from WM1552C/34b cells and from vector only control cells (WM1552C/VO), and the relative abundances of all coding and non-coding RNA were analyzed by deep sequencing. Sequence reads were mapped to the latest version of the human genome sequence (hg19) using two mapping algorithms (see Methods), and differentially expressed genes were classified by Gene Ontology classification. The most differentially expressed genes could be characterized into the categories of “cytoskeletal remodeling” (both TGF- and WNT-dependant and independent) and “cell adhesion” pathway network modules (FIG. 18A).

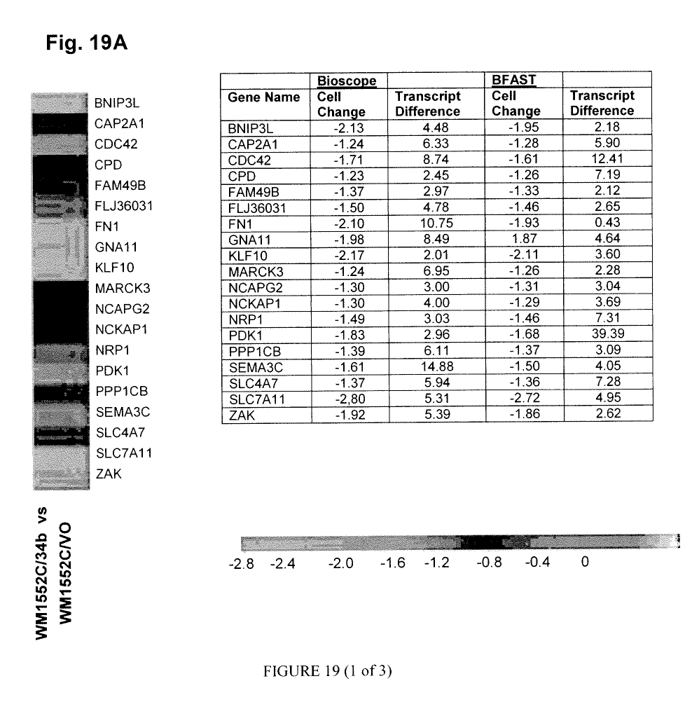

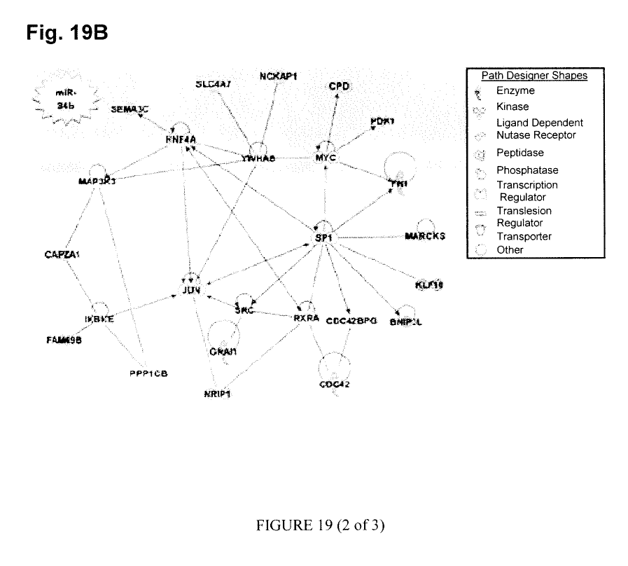

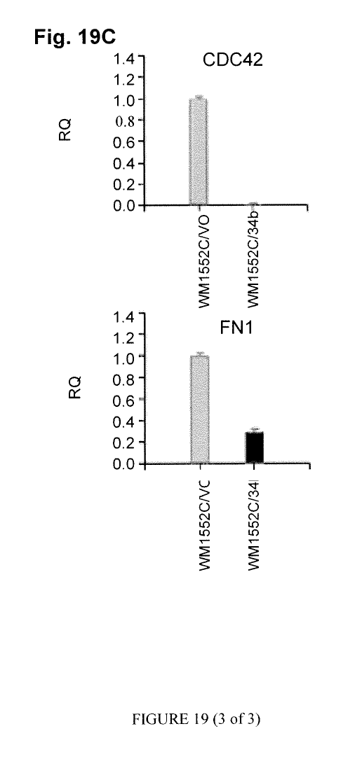

Out of a group of 19 computationally predicted target genes that were down-regulated in WM1552C/miR-34b cells (FIG. 19A), 15 are involved in cytoskeletal rearrangement, cell morphology, cell adhesion, cell motility, and invasion (FIG. 19B). Four of these gene targets (CAPZA1, CDC42, NCKAP1, and PPP1CB) were previously shown to regulate actin filament polymerization/reorganization (Aspenstrom, 1997; Eden et al., 2002; Maun et al., 1996; Tan et al., 2001). Down-regulation of two of these targets (CDC42 and FN1) was confirmed by quantitative RT-PCR analysis (FIG. 19C): CDC42 expression was 90% lower in WM1552C/34b cells and FN1 expression was nearly 70% lower, respectively, relative to vector only control cells.

In addition to the above genes, a number of differentially expressed mRNAs (34 down-regulated and 19 up-regulated) are shown in the FIG. 19B (see also Table 2). The majority of the corresponding genes are involved in the regulation of cell morphology, cell movement, and cell growth. A number of these genes (ALCAM, CAV1, DKK1, INHBA, MIA, MMP8, S100B, STC1, TGFBI, THBS2, TNS3, and WNT5A) have also been implicated in cell proliferation, migration, cell adhesion, invasion, and metastasis (Hawighorst et al., 2001; Liu et al.; Qian et al., 2009; Schneider et al., 2002; Seder et al., 2009). Interestingly, ALCAM, CAV1, DKK1, MIA, MMP8, S100B, and WNT5A have previously been shown to be involved in melanoma progression (Felicetti et al., 2009; Karnell et al., 1997; Kuphal et al., 2006; Palavalli et al., 2009; van Kempen et al., 2000; Weeraratna et al., 2002; Weilbach et al., 1990) and MIA and S100B are clinically utilized biomarkers for melanoma (Karnell et al., 1997; Weilbach et al., 1990). It is worth noting that CAV1, found to be down-regulated in WM1552C/34b cells, was previously identified as a target for miR-34b (Toyota et al., 2008). Two differentially regulated mRNAs (THBS2 and DKK1) identified by deep sequencing were validated using Taqman qRT-PCR analysis (FIG. 18D). THBS2 expression was nearly 1½-fold higher in WM1552C/34b cells than in WM1552C/VO cells, whereas DKK1 was nearly 10-fold lower in WM1552C/34b.

Next-generation RNA sequencing also revealed a number of miRNAs that were differentially expressed in the presence of miR-34b (FIG. 18C, Table 3). Several such miRNAs (miR-20b, -134, -140, and -199b) were previously reported to be involved in cancer progression (Chao et al., 2010; Guo et al., 2010; Lei et al., 2009; Song et al., 2009). miR-140 was further validated by qRT-PCR and its expression was nearly 3-fold higher in WM1552C/34b cells compared to WM1552C/VO cells. These results suggest the existence of a global network of miRNAs and coding genes which is perturbed by miR-34b in melanoma.

TABLE 3

| | |
|---|---|
| Bioscope | BFAST |

| | Fold | Transcript | Fold | Transcript |
|---|---|---|---|---|
| miRNA Name | Change | Difference | Change | Difference |
| MIR663 | 12.50 | 70.74 | 1.71 | 29.24 |
| MIR663B | 4.48 | 35.55 | 1.99 | 27.73 |
| MIR140 | 3.65 | 21.18 | 2.16 | 10.76 |
| MIR409 | 3.50 | 12.15 | 5.11 | 7.38 |
| MIR34B | 3.26 | 11.50 | 4.95 | 3.95 |
| MIR1312 | 3.16 | 9.85 | 1.58 | 2.73 |
| MIR154 | 3.14 | 6.63 | 1.83 | 3.01 |
| MIR134 | 3.02 | 6.50 | 1.80 | 1.42 |
| MIR20B | 2.78 | 6.06 | 1.50 | 5.24 |
| MIR494 | 2.72 | 5.38 | 2.08 | 1.75 |
| MIR199B | 2.68 | 5.06 | 1.67 | 3.68 |
| MIR660 | 2.65 | 4.70 | 3.11 | 5.07 |
| MIR149 | 2.59 | 4.50 | 1.91 | 5.56 |
| MIR453 | 2.44 | 4.29 | 2.93 | 2.27 |
| MIR432 | 2.38 | 4.17 | 1.78 | 3.76 |
| MIR495 | 2.35 | 4.14 | 2.94 | 3.47 |
| MIR410 | 2.30 | 3.86 | 2.01 | 2.38 |
| MIR301A | 2.17 | 3.86 | 1.62 | 2.25 |
| MIR431 | 1.90 | 3.63 | 1.75 | 3.13 |
| MIR496 | 1.90 | 3.37 | 1.60 | 2.25 |
| MIR487A | 1.83 | 2.96 | 2.06 | 1.56 |
| MIR1197 | 1.83 | 2.86 | 1.58 | 1.06 |
| MIR376A2 | 1.82 | 2.78 | 2.08 | 1.14 |
| MIR518A2 | 1.74 | 2.74 | 1.88 | 1.16 |
| MIR382 | 1.72 | 2.70 | 1.95 | 1.12 |
| MIR362 | 1.70 | 2.09 | 1.82 | 1.56 |
| MIR656 | 1.64 | 2.08 | 1.97 | 1.08 |
| MIR425 | 1.62 | 1.82 | 1.55 | 1.66 |
| MIR323 | 1.57 | 1.76 | 2.24 | 1.78 |
| MIR380 | 1.54 | 1.65 | 2.14 | 1.14 |
| MIR501 | 1.52 | 1.44 | 2.26 | 1.26 |
| MIR1205 | 1.51 | 1.32 | 1.72 | 1.06 |
| MIR181D | 1.51 | 1.32 | 1.55 | 1.22 |

### Example 7

### The Effect of miR-34B on Melanoma Cell Growth, Adhesion, Migration, and Invation

### Growth and Adhesion Assays

Exponentially growing cells were trypsinized and counted with the help of a Countess cell counter (Invitrogen/Life Technologies) and seeded at 10⁴ cells/cm² (0.1×10⁶ cells/35-mm petri dish), which was taken as the initial value (N₀) for all growth kinetics measurements. Cells were harvested at 24-hrs intervals up to 72 hrs by trypsinization. A minimum of 6 observations spread over 2 independent experiments were taken for determining the proliferation rate ‘pR’:

pR=Nₜ/N₀ (I)

Where N₀=number of cells at 0 h, and Nₜ=number of cells at time ‘t’. The mean doubling time of untreated cells was calculated from the exponential region of the growth curves, in this case between 24 hrs and 72 hrs.

Trypsinized cells were counted, and 50,000 cells per well were seeded into 96-well plates. At 5, 15, and 30 min after seeding, floating cells were aspirated by rinsing the wells with PBS. The remaining cells in the wells were fixed and stained with 0.5% crystal violet in 50% methanol. The stain in the fixed cells was dissolved with acetic acid, and cell density was determined spectrophotometrically by measuring absorbance at OD₅₆₀. Each sample was assayed in triplicate at each time point, and each experiment was repeated twice.

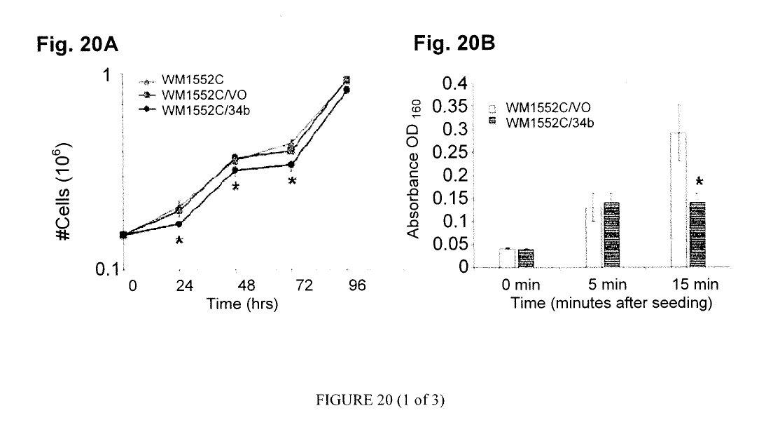

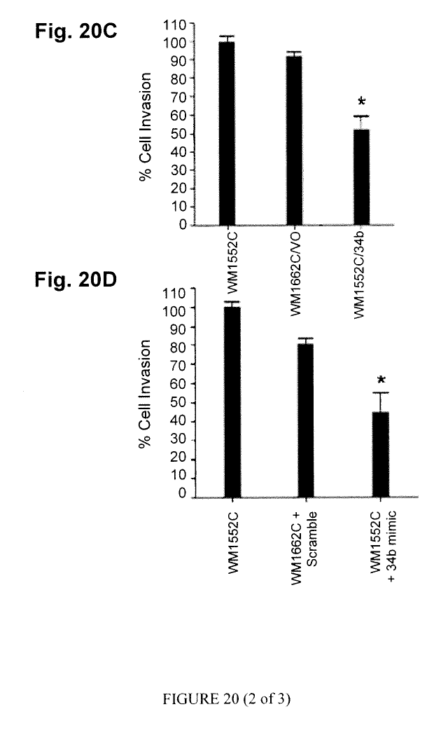

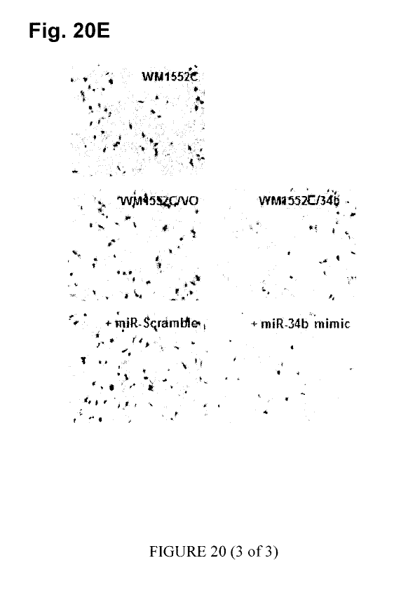

Since miR-34b expression is epigenetically regulated in certain classes of melanoma, its effects on melanoma cells were examined by cell biology assays. When artificially expressed, WM1552C/34b cells exhibited decreased proliferation (FIG. 20A) compared to control cells (WM1552C and WM1552C/VO). This decrease continued to progress until the end of the assay (96 hrs). WM1552C/34b cells also showed decreased cell adhesion compared to WM1552C/VO cells, apparent by as little as 15 minutes after plating the cells (FIG. 20B).

### Migration Assay

WM1552C/34b and WM1552C/VO cells were seeded on Mat Tek 1.5 mm tissue culture dishes until 90-95% confluent. Cell monolayers were then gently scratched with a pipette tip across the entire diameter of the dish and extensively rinsed with medium to remove all cellular debris. The surface area of the denuded surface was quantified immediately after wounding and again every 20 minutes for 24 hrs on the Nikon Bio Station IM. The extent of wound closure was determined by calculating the ratio of the surface area between the remaining wound edges for each time point to the surface area of the initial wound. These data were then expressed as the percentage of wound closure relative to the control conditions for each experiment. The surface area was calculated using NIS Elements software and performed in triplicate.

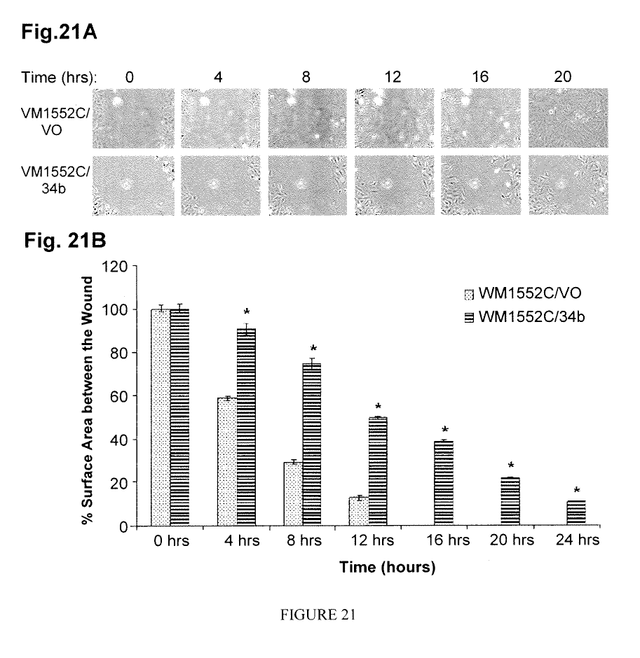

A wound-healing assay was performed to assess the effect of miR-34b on cell motility. A striking difference in the rates of motility was observed as early as 4 hrs after the initial scratch (FIGS. 21A and 21B). By the end of 8 hrs, the WM1552C/34b-expressing cells migrated less than half of the distance of WM1552C/VO cells. While WM1552C/VO cells closed the scratch by 16 hrs, WM1552C/34b cells took 24 hrs to close the scratch. These results suggest that the expression of miR-34b plays an important role in the motility of melanoma cells. Taken together, these results highlight the importance of miR-34b regulation in cell adhesion, invasion, and motility in human melanomas.

### Invasion Assay

BD BioCoat™ growth factor reduced insert plates (Matrigel™ Invasion Chamber 12 well plates) were prepared by rehydrating the BD Matrigel™ matrix coating in the inserts with 0.5 ml of culture medium for 2 hrs at 37° C. The rehydration solution was carefully removed from the inserts, 0.75 mL Complete Tu medium containing chemoattractant (1% FBS) was added to the lower wells of the plate, and 0.5 mL of cell suspension (1×10⁴ cells, in serum-free medium) was added to each insert well. WM1552C cells were transfected with mimic hsa-miR-34b as well as Negative Control#1 (Ambion) (miR-Scramble) at a concentration of 100 nM using siPORT NeoFX (Ambion). Invasion assay plates were incubated for 48 hrs at 37° C. Following incubation, the non-invading cells were removed by scrubbing the upper surface of the insert. The cells on the lower surface of the insert were stained with crystal violet and each trans-well membrane mounted on a microscopic slide for visualization and analysis. The slides were scanned using the Scanscope digital slide scanner, and the number of cells migrating was counted using Aperio software. Data are expressed as the percent invasion through the membrane relative to the migration through the control membrane, where percent invasion=Mean number of cells invading through the Matrigel insert membrane/Mean number of WM1552C (wild-type) cells migrating through membrane.

Finally, the effect of miR-34b on invasion was assessed. Both WM1552C/34b and vector only control cells were seeded into invasion chambers and the cells were allowed to migrate for 48 hrs. WM1552C/34b cells migrated significantly slower (46%) when compared to WM1552C and WM1552C/VO cells (FIGS. 21C and 21E). This observation was further confirmed by transient transfection of miR-34b mimics into melanoma cells. A similar reduction in cell invasion was observed and results are shown in FIGS. 21D and 21E. It should be noted that the reduction in cell invasion considerably exceeded the reduction in proliferation rate, suggesting that these two processes are independent of each other.

### Example 8

### The effect of miR-375 Ectopic Expression on Cellular Morphology, Proliferation, Migration, and Invasion in Melanoma Cells

### Cellular Morphology

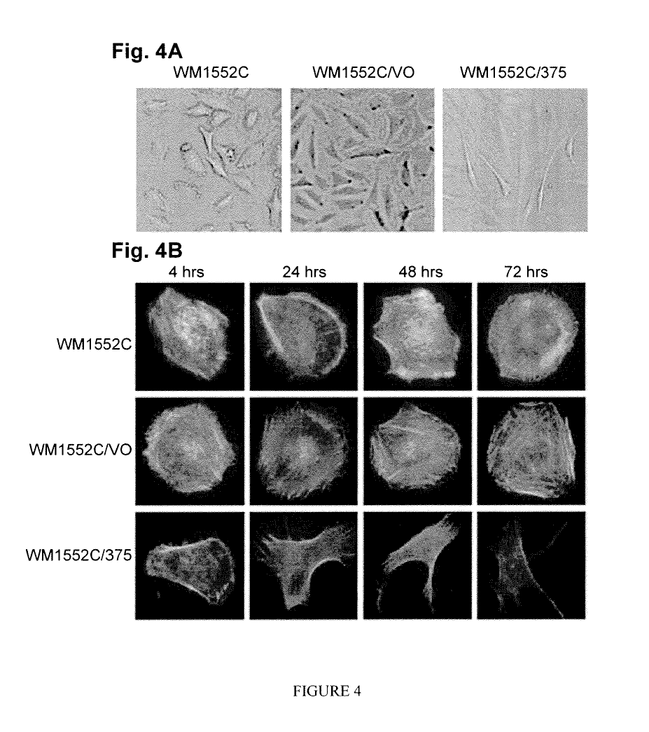

Changes in cellular morphology of stable miR-375-expressing melanoma cells were measured compared with “vector-only” and untransfected cells, as shown in FIG. 4. To construct a stable miR-375-expressing melanoma cell line, oligonucleotides complimentary to the has-miR-375 genomic sequences were constructed with miR-375 pre For (CCC CGC GAC GAG CCC C; SEQ ID NO:27) and miR-375 pre Rev (GCC TCA CGC GAG CCG AAC G; SEQ ID NO:28). These oligonucleotides contained HindIII and BamHI sites on their respective 5′ and 3′ ends and were amplified from melanocyte genomic DNA (Amplitaq Gold, Applied Biosystems/Life Technologies). The product was then TOPO-cloned into pCR4-TOPO (Invitrogen/Life Technologies) and the vector construct was sequenced. The pre-hsa-miR-375 fragment was sub-cloned into pcDNA4/H1 using the HindIII and BamHI sites to create pcDNA/H1/miR-375. 2.5×10⁵ WM1552C melanoma cells were seeded into a single well of a 6-well plate and transfected with 5 μg pcDNA4/H1/miR-275 or pcDNA4-H1 (vector only control) using Fugene 6 (Roche). The following day, cells were selected with 800 μg/mL Zeocin for the following 15 days. The surviving stable cells were then expanded and named WM1552C/375 and WM1552C/VO (vector only).

Live cells were imaged under normal culturing conditions by light microscope (Olympus IX71). The morphology time course was performed with cells growing exponentially, trypsinized and seeded with 2×10⁴ cells into 4-well Lab-Tek II chamber slides (Thermo Fisher Scientific), with one slide per time point. At the designated time points (4, 24, 48, and 72 hours), the slides were washed in PBS and fixed in 4% formaldehyde (in PBS). The slides were then labeled with DAPI and fluorescence-labeled phalloidin, then imaged on a Nikon Eclipse microscope.

Viewed by light microscopy as described above, the miR-375-expressing melanoma cells exhibited a more slender and elongated shape compared to the wild type and vector control cells, which were similar in appearance, as shown in FIG. 4(A). These stably-expressing cells were also examined by fluorescence microscopy after staining with FITC-labeled phalloidin to visualize filamentous actin. A background of phosphor-cortactin staining can be seen as a red color in some cells. FIG. 4(B) shows that the morphological changes in miR-375-expressing cells were time-dependent, with changes detectable within four hours after trypsinization and reseeding, and elongated filapodia-like extensions visible by 24 hours. These changes appeared complete by 72 hours. By comparison, both untransfected WM1552C and WM1552/VO cells acquired their characteristic rounded shape by four hours, and little change in morphology was seen thereafter, as shown in FIG. 4(B).

### Proliferation Assay

Exponentially growing cells were trypsinized and counted with the help of a Countess cell counter (Invitrogen/Life Technologies) and seeded at 10⁴ cells/cm² (0.1×10⁶ cells/35-mm petri dish), which was taken as the initial value (N₀) for all growth kinetics measurements. Cells were harvested at 24-hrs intervals up to 72 hrs by trypsinization. A minimum of 6 observations spread over 2 independent experiments were taken for determining the proliferation rate ‘pR’:

pR=Nₜ/N₀ (I)

where N₀=number of cells at 0 h, and Nₜ=number of cells at time ‘t’. The mean doubling time of untreated cells was calculated from the exponential region of the growth curves, in this case between 24 hrs and 72 hrs.

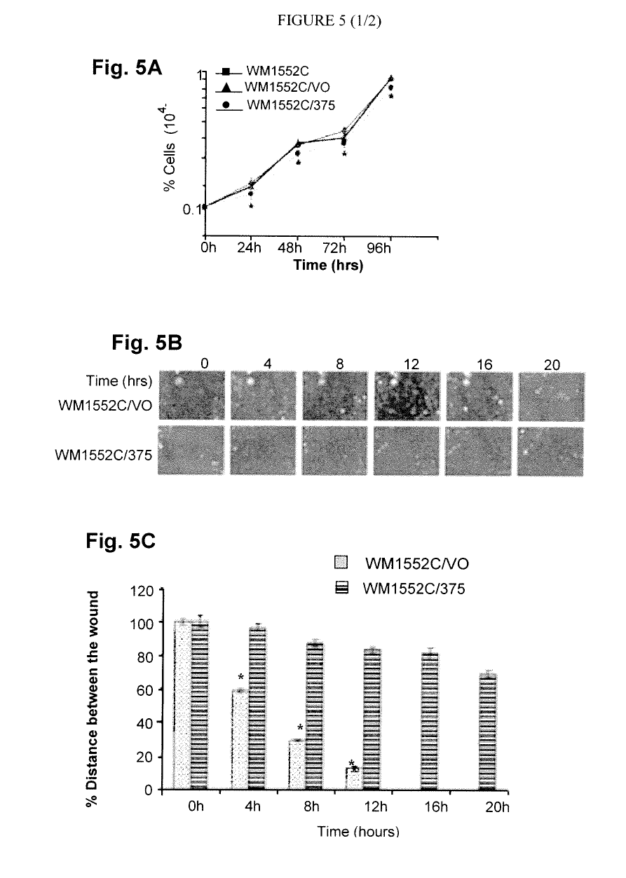

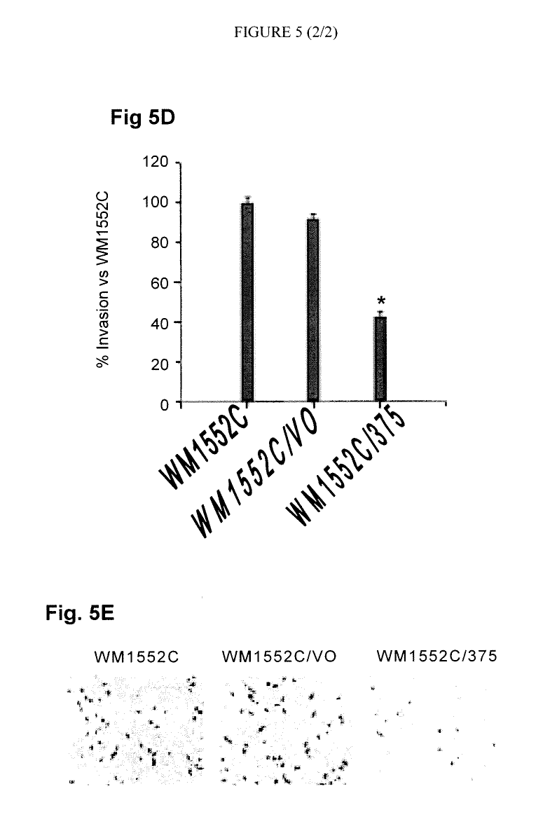

As shown in FIG. 5(A), proliferation of WM1552C/375 cells was reduced approximately 10 to 15% compared to that of WM1552C or WM1552C/VO cells, and the decrease was sustained over the 96 hour assay.

### Motility Assay

The in vitro motility of transfectants was measured in a wound-healing assay conducted as follows. Mat Tek 1.5 mm tissue culture dishes were seeded with WM1552C/375 or WM1552C/VO cells until 90-95% confluent. The cell monolayers were gently scratched with a pipette tip across the diameter of the dish and rinsed extensively with medium to remove cellular debris. The surface area of the scratch was quantified immediately after wounding and then again every 20 minutes for 24 hours on the Nikon Bio Station IM. The extent of wound closure was established by calculating the ratio between the surface area of the wound for each time point versus the surface area of the initial wound. These data were then expressed as the percentage of wound closure relative to the control conditions. The surface area was determined using NIS Elements software and performed in triplicates.

A striking difference was noted in the rates of motility of cells expressing miR-375 as early as four hours after initiation of the assay, as shown in FIGS. 5(B) and (C). By 12 hours of incubation, wound healing was nearly complete in cultures of WM1552C/VO cells, whereas migration of WM1552C/375 cells was dramatically reduced with less than 30% wound closure by 20 hours.

### Invasion Assay

BD BioCoat™ growth factor reduced insert plates (Matrigel™ Invasion Chamber 12 well plates) were prepared by rehydrating the BD Matrigel™ matrix coating in the inserts with 0.5 ml of culture medium for 2 hrs at 37° C. The rehydration solution was carefully removed from the inserts, 0.75 mL Complete Tu medium containing chemoattractant (1% FBS) was added to the lower wells of the plate, and 0.5 mL of cell suspension (1×10⁴ cells, in serum-free medium) was added to each insert well. Invasion assay plates were incubated for 48 hrs at 37° C. Following incubation, the non-invading cells were removed by scrubbing the upper surface of the insert. The cells on the lower surface of the insert were stained with crystal violet and each trans-well membrane mounted on a microscopic slide for visualization and analysis. The slides were scanned using the Scanscope digital slide scanner, and the number of cells migrating was counted using Aperio software. Data are expressed as the percent invasion through the membrane relative to the migration through the control membrane, where percent invasion=Mean number of cells invading through the Matrigel insert membrane/Mean number of WM1552C (wild-type) cells migrating through membrane.

The results as shown in FIGS. 5(D) and (E) indicate that WM1552C/375 cells migrated at less than 50% the frequency of either wild type or vector-expressing cells. The reduction in cell invasion exhibited by miR-375-expressing cells is much greater than the observed effect on cell proliferation as shown in FIG. 5(A), indicating that these two effect of miR-375 expression occur independently. Therefore, our results demonstrate that ectopic expression of miR-375 has deleterious effects on melanoma cell morphology, motility, proliferation, and invasion.

### Example 9

### The Global Gene Expression Profiling of Stable miR-375-Expressing Melanoma Cell Lines by Next-Generation Sequencing Reveals Putative Target Genes

To further probe the pathological relevance of miR-375 on malignant melanoma, total RNA was isolated from WM1552C/375 or WM1552C/VO cells as described below. Total RNA was reverse transcribed into cDNA, and analyzed by next-generation sequencing. The sequences were mapped to the human genome and mined for differences in RNA species expression.

Isolation of total RNA was performed using Trizol (Invitrogen/Life Technologies), and further fragmented by RNas III digestion. The purified RNA was ligated to an adapter and reverse transcribed by priming the adapter, with the resulting cDNA amplified and separated using a 6% urea gel. SYBR Gold dye was used for sequencing. The resulting sequence libraries (approximately 150-200 bp size fragments) were further amplified by a bead-based emulsion PCR designed to individually isolate a single bead/cDNA molecule. This allows for massively parallel amplification of monoclonal DNA species. The RNA-seq experiments then deposited approximately 90 million beads per sample onto a glass slide and analyzed the results using ligation-based sequencing technology. The data was then mapped to the human genome using Bioscope and BFAST software packages and was analyzed at the Burnham Institute Bioinformatics core facility.

Mapping of the SOLiD sequence data was performed using the standard pipeline of Blat-like Fast Accurate Search Tool (BFAST; Horner et al., 2009) against the human genome (hg19; Rhead et al., 2010). The BFAST algorithm first creates indices of the human genome using different masks. All ten masks suggested in the BFAST manual were used in this assay. It then hashes the reads to a number of genomic locations based on the indices and performs detailed alignments between the reads and the genomic sequence at the hashed locations. The final output of BFAST contains the mapped genomic locations of each read, and the qualities (represented via alignment scores) of the mappings.

The sequential analysis of the expression level of the genes is based on the mapping results. The process successfully mapped approximately 63% of the reads to the reference genome, and among those mapped reads, approximately 89% are uniquely mapped. Only uniquely mapped reads were considered to eliminate possible noise. For each gene (the RefSeq annotation for hg19), a score was calculated as reads per kilobase of sequence per million reads (RPKM; Mortazavi et al., 2008). The predefined mapping protocol for SOLiD sequencing on BioScope 1.0 was also used. The output was then fed into the Partek Genomics Suite for analysis. Again, an RPKM score was computed for each gene.

The results of the two analysis pipelines were compared to arrive at a consensus. Fold-change was computed for each gene as a ratio of the WM1552C/375 RPKM over the WM1552C/VO RPKM. For this calculation, a pseudo-count of 1.0 was used to overcome divide by zero errors and normalize the data at low RPKM values. A list of genes was produced such that both programs agreed on the regulation direction (i.e. upregulation or downregulation). The order of the genes was then directed by the corroborated fold change.

Expression levels of the target genes of miR-375 were then assessed. The target gene list was produced using TargetScan 5.1 as described in Friedman et al., 2009, which considers and ranks targets based on site type, 3′ pairing, local U/A concentration and position, with sequential verification on multiple species. The miR-375 sequence from miRBase was obtained (Griffiths-Jones et al., 2008), and the multiple sequence alignments (MSA) of 43 vertebrate assemblies to the Human Genome (hg18) was acquired from the UCSC Genome Browser. Based on the corresponding RefSeq annotation, the 3′UTR MSA for each gene was extracted. The TargetScan algorithm was applied on every species of the 3′UTR MSA for each gene. A comprehensive score for each 3′UTR MSA was compiled, which summed the TargetScan score of all species. The genes were then ranked according to the comprehensive score, and the top 200 ranked genes were selected as the candidate targets of miR-375.

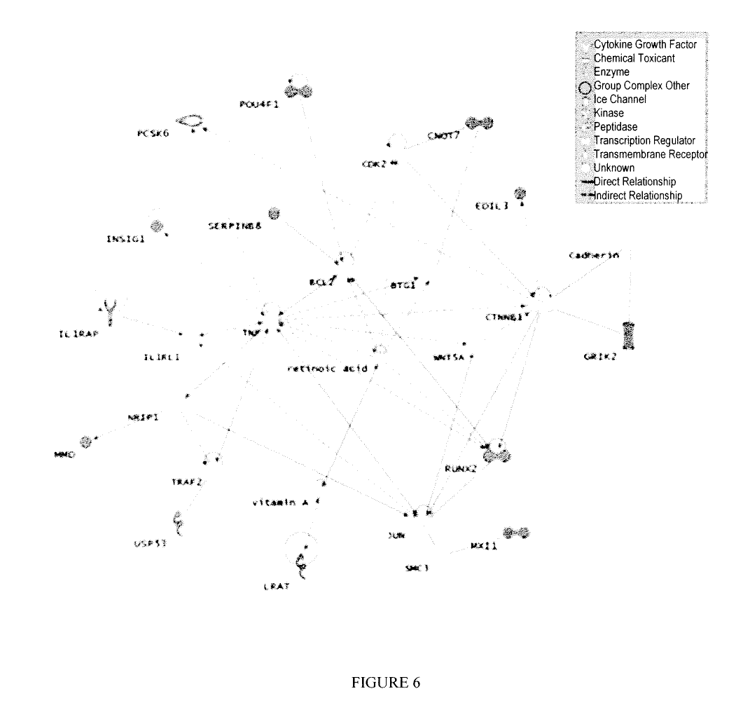

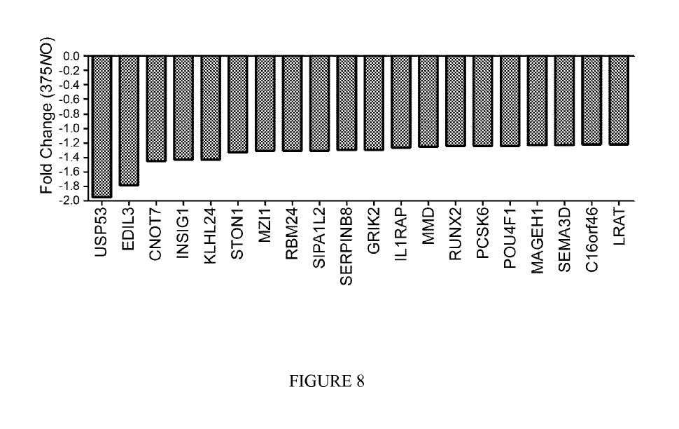

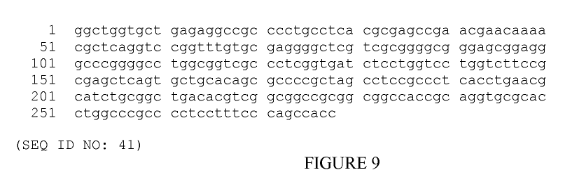

Twenty candidate target genes were identified as highly differentially expressed, as shown in FIG. 8. Systems level pathway mapping (Ingenuity pathway maps) revealed that 13 of the 20 candidate genes mapped to a single network regulating cellular and connective tissue development and function, as shown in FIG. 6, indicating that miR-375 regulates genes associated with cellular morphology and tissue networking.

### Example 10

### Methyl Binding Domain Protein 2 (MBD2) Enriched DNA Specifically Covers Regions with High Methyl-CpG Content in Human Melanoma Cell Lines and Melanocytes

In order to identify additional globally methylated CpG islands in the human genome, MBD2 was used to enrich highly methylated CpG islands in human melanoma cell lines and melanocytes, which were then deep-sequenced using the SOLiD-4.0 platform. Since methylated cytosine comprises only a small fraction of the human genome, the enrichment of highly methylated sequences prior to deep sequencing is more cost-effective than whole-genome bisulphate sequencing (Bis-seq). Therefore, MethylMiner™ (Lifetech, Invitrogen) was used to enrich the methlated regions in the melanoma genome.

Six melanoma cell lines, A375 (Stage IV, ATCC No. CRL-1619), HT144 (Stage IV, ATCC No. HTB-63), RPMI-7951 (stage IV, ATCC No. HTB-66), SKMEL2 (stage IV, ATCC No. HTB-68), WM793B (Stage I, ATCC No. CRL-2806), WM1552C (stage III, ATCC No. CRL-2808), as well as melanocyte cell line HEM-I (ScienCell, Catalog No. 2200; growth conditions: MelM media containing MelGS supplements with 0.5% FBS and pen/strep) were used for the CpG island enrichment. All melanoma cell lines were grown in Complete Tu media: a 4:1 mixture of MCDB-153 with 1.5 g/L sodium bicarbonate and Leibovitz L-15 with 2 mM L-glutamine, plus 2% FBS and 1.68 mM CaCl₂. All clinical samples were acquired from frozen samples graciously donated by Dr. James Goydos of Robert Wood Johnson Medical School.

Genomic DNA from the cell lines listed above was fragmented to 50-400 bp (mean ˜250 bp) using Covaris™ S2 systems (Woburn, Mass.), and 10 μg of DNA was subjected to MBD2-protein capture according to the manufacturer's protocol. Varying concentration of NaCl enrichment buffer were used (200 nM, 350 nM, 450 nM, 600 nM, 1 M, and 2M) for single fraction elution in A375 cells. The higher salt concentration (2M) produced tighter enrichment of CpG islands in the MBD2 pull-down samples, and was therefore used for all subsequent sample enrichments. The methylated DNA fragments recovered from the first two incubations of 2M salt concentration were pooled, then ethanol precipitated and resuspended. The DNa and samples of DNA that did not go through the MBD2 pull-down (whole genome, unenriched) were used for fragment library construction, which included a gel-based size selection step to obtain a mean insert length of approximately 150 bp.

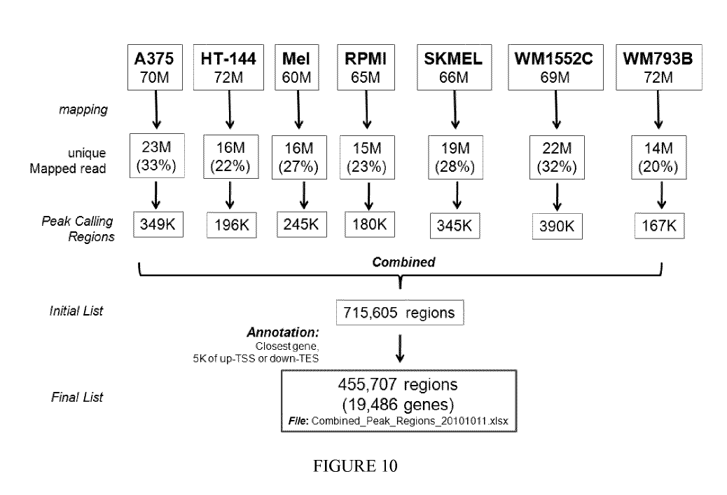

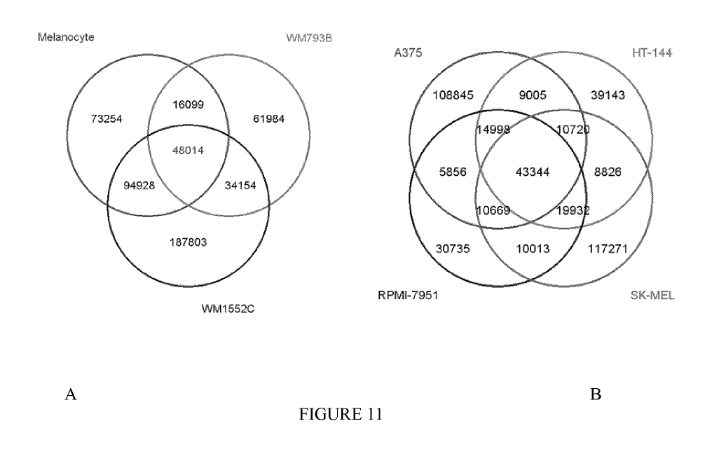

To characterize the sequences captured by MBD2, the enriched DNA fragments were subjected to SOLiD-4.0 DNA sequencing. First, raw data (DNA sequences) were examined for their sequence quality. Sequences were then mapped to reference human genome (Hg19). MBD2-enriched regions that contain highly methylated CpG islands were further analyzed for cell-type specific methylation patterns. In particular, cell type specific methylation patterns from A375, HT-144, RPMI-7951, SK-MEL2, WM793B, WM1552, and melanocytes were identified for further analysis, as shown in FIG. 10. FIG. 10 demonstrates that the peak calling regions were 349K, 196K, 180K, 345K, 390K, 167K, and 245K, respectively. The total number of initial peak callings (715K) was further characterized and mapped to a region of 5 Kb upstream of the transcriptional start site (TSS) and/or downstream of transcription end sites (TES) of coding genes. In total, 455K regions were aligned to 19,486 genes. FIG. 11A demonstrates highly methylated CPG islands common and unique to melanocytes, WM793B and WM1552C. Likewise, in FIG. 11B, A375, HT144, RPMI, and SKMEL-2 were further compared to identify common and different genes.

To remove DNA methylation, WM1552C cells were treated with 5 AzadC. The cells were plated into 75-cm³ flasks at a concentration of 5×10⁵ cells per flask. Each flask was treated with 10 ng/mL 5-Aza-dC with a complimentary flask left untreated. The cells were washed daily with PBS, given fresh media, and treated. Treatment persisted for 5 days, after which the cells were washed again with PBS, trypsinized and harvested, and centrifuged at 1200 rpm for five minutes. All cell pellets were then prepared using the Trizol protocol (Invitrogen/Life Technologies) for total RNA, and quantified using the ND-1000 spectrophotometer (NanoDrop). The removal of methylation was verified by sequencing enriched DNA with MBD2.

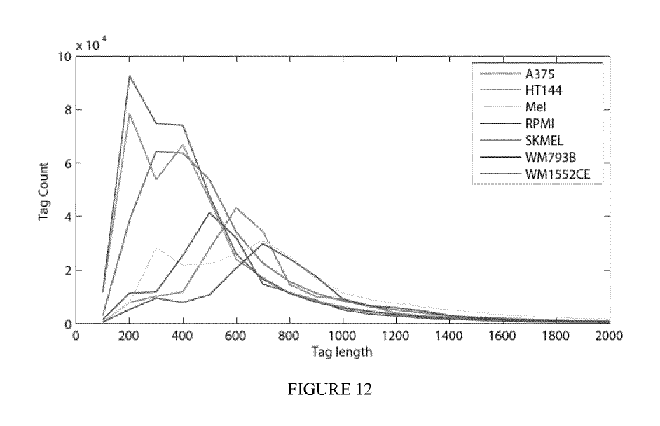

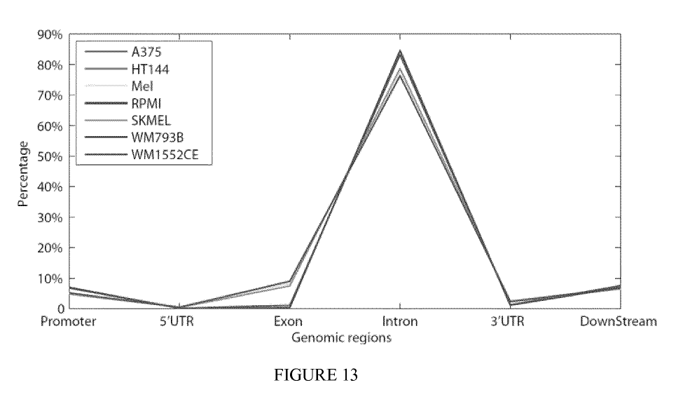

The MACS algorithm was then used to detect sequence peaks and reported as RPKM value. For the analysis process, genomic regions are separated into seven categories: promoter region, 5′UTR, exon, intron, 3′UTR, downstream region, and intergenic region. The promoter region is defined as 3 kb upstream of the transcription start site, and the downstream region is defined as 3 kb following the stop sign. Some of the downstream sequence regions overlap with 3′UTR of a given gene. The sequences that do not overlap the promoter region, 5′UTR, exon, intron, 3′UTR, and downstream region, are classified as intergenic regions. Genome annotations are downloaded from UCSC genome browser. Next, BEDTools were applied to find the intersection between peaks and different genomic regions. For peaks that belong to multiple genomic regions, ties were broken by the length of the overlapped regions. For example, if a peak overlaps 300 bp with a gene's promoter region and 400 bp with another gene's intronic region, the peak is assigned as an intronic region instead of a promoter region. Next, each peak was assigned a specific genomics region. FIG. 12 illustrates the global CpG island tag length and tag count in melanoma cell lines and melanocytes. According to FIG. 12, the highest numbers of tag counts were seen between 100 bp to 400 bp tag lengths, but methylated CpG islands were spread between 100 bp to 800 bp. Tag counts of all melanoma cell lines and melanocytes were normalized to stage III melanoma cell line WM1552C input (non-enriched) DNA. The highest number of tag counts were obtained from stage IV melanoma cells A375, SKMEL-2, and stage III cell line WM1552C. Melanocytes showed relatively low levels of CpG methylation compared to melanoma cells. FIG. 13 shows the percentage of methylation in genomics regions, and demonstrates that the highest region of methylation in the genome was observed in the intronic regions.

To identify and validate differentially methylated (DMR) CpG islands in the regulatory regions of coding genes in melanoma, the promoter region was first identified. WM1552C cells were used herein as exemplary of the methylation of melanocytes. DNA was first isolated from MBD2 enriched and non-enriched and also cells treated with 5AzadC and then enriched with MBD2 for subsequent DNA sequencing. Sixty-eight million reads were generated from WM1552C, MBD2-enriched DNA, and eighty-eight million reads were obtained from DNA treated with 5AzadC followed by the MBD2 enrichment. As for the control, seventy-four million reads were obtained from input DNA (no enrichment). MACS: 1.0e-10 for methylation peak-calling algorithm was used to identify 160K peaks that were highly methylated and uniquely mapped compared to input and 5azadC treated controls.

To determine the number of methylated regions located on the promoter regions of coding genes, the methylated regions close to the coding genes, i.e., 2 Kb upstream or 1 Kb downstream of the TSS, were mapped. This process generated 7,590 regions that are located in the promoter region of 5,637 genes. This set is narrowed by incorporating nearby CpG island information, clear annotated promoter and 1 Kb upstream of the TSS. Finally, 583 highly methylated regions were identified in 581 genes. Next, this subset was characterized according to the relevance to melanoma, and the level of CpG island methylation intensities. CpG island methylation information is listed in Table 2. Highly methylated promoter regions of coding genes were further associated with their corresponding RNA-seq and microarray expression data, resulting in the connection between promoter methylation and corresponding gene expression being established. Several CpG promoter methylated genes were further validated by bisulphate DNA sequencing as described below:

Samples of genomic DNA were prepared from 107 cells of each cell line. Samples were harvested by trypsinization, spun down at low speed (1200 rpm), and washed with phosphate-buffered saline (PBS). Cell pellets were then processed using the QiaAmp DNA mini kit (Qiagen). Genomic DNA was harvested from 25 mg of frozen patient samples and isolated by incubation with proteinase K at 55° C. overnight. Samples were subsequently purified with the QiaAmp DNA mini kit. All sample yields were determined using a ND-1000 spectrophotometer (Nanodrop). 500 ng of each sample of genomic DNA was then treated with sodium bisulfite overnight and purified and eluted with the EZ DNA methylation kit (Zymo Research).

Bisulphite PCR was performed using genomic eluate with the following primer combinations: PCSK1 Meth For (GGG TAG ATA AGG AGT AGA TTT AAT TGA TTT TAG; SEQ ID NO:29) and PCSK1 Meth Rev (CTC TAA ACC ACT CCT AAC TCC TA ATTA CTC; SEQ ID NO:30) to amplify a 253 bp region, CYP1B1 Meth For 2 (GGA GTT GAT TTT TTG GAG AAA TGG T; SEQ ID NO:31) and CYP1B1 Meth Rev (CTT ACC CTA AAC AAA AAT CCC AAT TCC TTC; SEQ ID NO:32) to amplify a 301 bp region, QPCT Meth For (GGG TTT AGA AGT TTG TGT TTG TTA TTT AGG G; SEQ ID NO:33) and QPCT Meth Rev 3 (CCC AAA ACA AAA CGA CCA CCA ACA ACA AC; SEQ ID NO:34) to amplify a 243 bp region, TERC Meth For (GGG TTA GTA GTT GAT ATT TTT TGT TTG TTT TAG; SEQ ID NO:35) and TERC Meth Rev 2 (CCT AAA AAA AAT AAT AAC CAT TTT TTA TCT AAC CC; SEQ ID NO:36) to amplify a 107 bp region, Kit Meth For (TGG GAG GAG GGG TTG TTG TT; SEQ ID NO:37) and Kit Meth Rev 2 (TAC CAC CCT CCC AAA CAC AAA CTT C; SEQ ID NO:38) to amplify a 210 bp region, and SNORD 10 Meth For (GGT GGT TAT GGT ATT AGG AGA TTA TAT GGG; SEQ ID NO:39) and SNORD 10 Meth Rev (CTC TTC CCC CAA AAA AAA ACC AAC ATC C; SEQ ID NO:40) to amplify a 219 bp region. All PCR was performed using a 2-minute hot start at 95° C., followed by 35-40 cycles at 94° C. for 20 seconds, 54° C. for 20 seconds, and 72° C. for 45 seconds, ending with a 2-minute extension at 72° C. using GoTaq Green (Promega). All PCR products were gel purified from agarose gels using the QiaQuick gel extraction kit (Qiagen) and subsequently cloned into the PCR4-TOPO vector (Invitrogen/Life Technologies). Six clones for each methylation region were sequenced (Retrogen) using the primers M13 for (−20) and M13 Rev. Sequences were then aligned and analyzed using Vector NTi AlignX (Invitrogen/Life Technologies).

FIG. 14A-F illustrates the results of the validation of promoter DNA methylation by bisulphate sequencing of coding genes PCSK1, CYP1B1, QPCT, and cKIT.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

The following references are cited herein to aid in a more complete understanding of the present invention:
1. Houghton, A. N., and Polsky, D. (2002) Focus on melanoma, Cancer Cell 2, 275-278.
2. Hoek, K., Rimm, D. L., Williams, K. R., Zhao, H., Ariyan, S., Lin, A., Kluger, H. M., Berger, A. J., Cheng, E., Trombetta, E. S., Wu, T., Niinobe, M., Yoshikawa, K., Hannigan, G. E., and Halaban, R. (2004) Expression profiling reveals novel pathways in the transformation of melanocytes to melanomas, Cancer Res 64, 5270-5282.
3. Zhang, L., Huang, J., Yang, N., Greshock, J., Megraw, M. S., Giannakakis, A., Liang, S., Naylor, T. L., Barchetti, A., Ward, M. R., Yao, G., Medina, A., O'Brien-Jenkins, A., Katsaros, D., Hatzigeorgiou, A., Gimotty, P. A., Weber, B. L., and Coukos, G. (2006) microRNAs exhibit high frequency genomic alterations in human cancer, Proc Natl Acad Sci USA 103, 9136-9141.
4. Gaur, A., Jewell, D. A., Liang, Y., Ridzon, D., Moore, J. H., Chen, C., Ambros, V. R., and Israel, M. A. (2007) Characterization of microRNA expression levels and their biological correlates in human cancer cell lines, Cancer Res 67, 2456-2468.
5. Fabbri, M., Garzon, R., Cimmino, A., Liu, Z., Zanesi, N., Callegari, E., Liu, S., Alder, H., Costinean, S., Fernandez-Cymering, C., Volinia, S., Guler, G., Morrison, C. D., Chan, K. K., Marcucci, G., Calin, G. A., Huebner, K., and Croce, C. M. (2007) MicroRNA-29 family reverts aberrant methylation in lung cancer by targeting DNA methyltransferases 3A and 3B, Proc Natl Acad Sci USA 104, 15805-15810.
6. Fazi, F., Racanicchi, S., Zardo, G., Starnes, L. M., Mancini, M., Travaglini, L., Diverio, D., Ammatuna, E., Cimino, G., Lo-Coco, F., Grignani, F., and Nervi, C. (2007) Epigenetic silencing of the myelopoiesis regulator microRNA-223 bp the AML1/ETO oncoprotein, Cancer Cell 12, 457-466.
7. Grady, W. M., Parkin, R. K., Mitchell, P. S., Lee, J. H., Kim, Y. H., Tsuchiya, K. D., Washington, M. K., Paraskeva, C., Willson, J. K., Kaz, A. M., Kroh, E. M., Allen, A., Fritz, B. R., Markowitz, S. D., and Tewari, M. (2008) Epigenetic silencing of the intronic microRNA hsa-miR-342 and its host gene EVL in colorectal cancer, Oncogene.
8. Lehmann, U., Hasemeier, B., Christgen, M., Muller, M., Romermann, D., Langer, F., and Kreipe, H. (2008) Epigenetic inactivation of microRNA gene hsa-mir-9-1 in human breast cancer, J Pathol 214, 17-24.
9. Mueller, D. W., Rehli, M., and Bosserhoff, A. K. (2009) miRNA Expression Profiling in Melanocytes and Melanoma Cell Lines Reveals miRNAs Associated with Formation and Progression of Malignant Melanoma, J Invest Dermatol.
10. Shenouda, S. K., and Alahari, S. K. (2009) MicroRNA function in cancer: oncogene or a tumor suppressor?, Cancer Metastasis Rev 28, 369-378.
11. Aqeilan, R. I., Calin, G. A., and Croce, C. M. miR-15a and miR-16-1 in cancer: discovery, function and future perspectives, Cell Death Differ 17, 215-220.
12. Lynam-Lennon, N., Maher, S. G., and Reynolds, J. V. (2009) The roles of microRNA in cancer and apoptosis, Biol Rev Camb Philos Soc 84, 55-71.
13. Schmittgen, T. D. (2008) Regulation of microRNA processing in development, differentiation and cancer, J Cell Mol Med 12, 1811-1819.
14. Chen, A., Luo, M., Yuan, G., Yu, J., Deng, T., Zhang, L., Zhou, Y., Mitchelson, K., and Cheng, J. (2008) Complementary analysis of microRNA and mRNA expression during phorbol 12-myristate 13-acetate (TPA)-induced differentiation of HL-60 cells, Biotechnol Lett 30, 2045-2052.
15. Igoucheva, O., and Alexeev, V. (2009) MicroRNA-dependent regulation of cKit in cutaneous melanoma, Biochem Biophys Res Commun 379, 790-794.
16. Baugh, L. R., Demodena, J., and Sternberg, P. W. (2009) RNA Pol II accumulates at promoters of growth genes during developmental arrest, Science 324, 92-94.
17. Felicetti, F., Errico, M. C., Bottero, L., Segnalini, P., Stoppacciaro, A., Biffoni, M. Felli, N., Mattia, G., Petrini, M., Colombo, M. P., Peschle, C., and Care, A. (2008) The promyelocytic leukemia zinc finger-microRNA-221/-222 pathway controls melanoma progression through multiple oncogenic mechanisms, Cancer Res 68, 2745-2754.
18. Schultz, J., Lorenz, P., Gross, G., Ibrahim, S., and Kunz, M. (2008) MicroRNA let-7b targets important cell cycle molecules in malignant melanoma cells and interferes with anchorage-independent growth, Cell Res 18, 549-557.
19. Bemis, L. T., Chen, R., Amato, C. M., Classen, E. H., Robinson, S. E., Coffey, D. G., Erickson, P. F., Shellman, Y. G., and Robinson, W. A. (2008) MicroRNA-137 targets microphthalmia-associated transcription factor in melanoma cell lines, Cancer Res 68, 1362-1368.
20. Chen, C. Z. (2005) MicroRNAs as oncogenes and tumor suppressors, N Engl J Med 353, 1768-1771.
21. Dalmay, T., and Edwards, D. R. (2006) MicroRNAs and the hallmarks of cancer, Oncogene 25, 6170-6175.
22. Esquela-Kerscher, A., and Slack, F. J. (2006) Oncomirs—microRNAs with a role in cancer, Nat Rev Cancer 6, 259-269.
23. Perera, R. J., and Ray, A. (2007) MicroRNAs in the search for understanding human diseases, BioDrugs 21, 97-104.
24. Kent, O. A., and Mendell, J. T. (2006) A small piece in the cancer puzzle: microRNAs as tumor suppressors and oncogenes, Oncogene 25, 6188-6196.
25. Zhang, B., Pan, X., Cobb, G. P., and Anderson, T. A. (2006) microRNAs as oncogenes and tumor suppressors, Dev Biol.
26. Hammond, S. M. (2006) MicroRNAs as oncogenes, Curr Opin Genet Dev 16, 4-9.
27. Mazar, J., DeYoung, K., Khaitan, D., Meister, E., Almodovar, A., Goydos, J., Ray, A., and Perera, R. J. (2010) The regulation of miRNA-211 expression and its role in melanoma cell invasiveness, PLoS One 5, e13779.
28. Felicetti, F., Errico, M. C., Segnalini, P., Mattia, G., and Care, A. (2008) MicroRNA-221 and -222 pathway controls melanoma progression, Expert Rev Anticancer Ther 8, 1759-1765.
29. Segura, M. F., Hanniford, D., Menendez, S., Reavie, L., Zou, X., Alvarez-Diaz, S., Zakrzewski, J., Blochin, E., Rose, A., Bogunovic, D., Polsky, D., Wei, J., Lee, P., Belitskaya Levy, I., Bhardwaj, N., Osman, I., and Hernando, E. (2009) Aberrant miR-182 expression promotes melanoma metastasis by repressing FOXO3 and microphthalmia-associated transcription factor, Proc Natl Acad Sci USA 106, 1814-1819.
30. Levy, C., Khaled, M., Iliopoulos, D., Janas, M. M., Schubert, S., Pinner, S., Chen, P. H., Li, S., Fletcher, A. L., Yokoyama, S., Scott, K. L., Garraway, L. A., Song, J. S., Granter, S. R., Turley, S. J., Fisher, D. E., and Novina, C. D. (2010) Intronic miR-211 assumes the tumor suppressive function of its host gene in melanoma, Mol Cell 40, 841-849.
31. Saini, H. K., Griffiths-Jones, S., and Enright, A. J. (2007) Genomic analysis of human microRNA transcripts, Proc Natl Acad Sci USA 104, 17719-17724.
32. de Souza Rocha Simonini, P., Breiling, A., Gupta, N., Malekpour, M., Youns, M., Omranipour, R., Malekpour, F., Volinia, S., Croce, C. M., Najmabadi, H., Diederichs, S., Sahin, O., Mayer, D., Lyko, F., Hoheisel, J. D., and Riazalhosseini, Y. (2010) Epigenetically deregulated microRNA-375 is involved in a positive feedback loop with estrogen receptor alpha in breast cancer cells, Cancer Res 70, 9175-9184.
33. Tsukamoto, Y., Nakada, C., Noguchi, T., Tanigawa, M., Nguyen, L. T., Uchida, T., Hijiya, N., Matsuura, K., Fujioka, T., Seto, M., and Moriyama, M. (2010) MicroRNA-375 is downregulated in gastric carcinomas and regulates cell survival by targeting PDK1 and 14-3-3zeta, Cancer Res 70, 2339-2349.
34. Horner, N., Merriman, B., and Nelson, S. F. (2009) BFAST: an alignment tool for large scale genome resequencing, PLoS One 4, e7767.
35. Rhead, B., Karolchik, D., Kuhn, R. M., Hinrichs, A. S., Zweig, A. S., Fujita, P. A., Diekhans, M., Smith, K. E., Rosenbloom, K. R., Raney, B. J., Pohl, A., Pheasant, M., Meyer, L. R., Learned, K., Hsu, F., Hillman-Jackson, J., Harte, R. A., Giardine, B., Dreszer, T. R., Clawson, H., Barber, G. P., Haussler, D., and Kent, W. J. The UCSC Genome Browser database: update 2010, Nucleic Acids Res 38, D613-619.
36. Mortazavi, A., Williams, B. A., McCue, K., Schaeffer, L., and Wold, B. (2008) Mapping and quantifying mammalian transcriptomes by RNA-Seq, Nat Methods 5, 621-628.
37. Friedman, R. C., Farh, K. K., Burge, C. B., and Bartel, D. P. (2009) Most mammalian mRNAs are conserved targets of microRNAs, Genome Res 19, 92-105.
38. Griffiths-Jones, S., Saini, H. K., van Dongen, S., and Enright, A. J. (2008) miRBase: tools for microRNA genomics, Nucleic Acids Res 36, D154-158

What is claimed is:

1. A method for diagnosing melanoma in a subject suspected of having melanoma comprising:
(i) assessing the level of DNA methylation in a 5′ upstream CpG island of an epigenetically regulated differentially expressed coding or non-coding gene in a biological sample obtained from the subject; and
(ii) determining whether the assessed level indicates hypermethylation;
wherein hypermethylation indicates that the subject has melanoma, and the absence of hypermethylation indicates that the subject does not have melanoma.

2. The method of claim 1, wherein the gene is selected from the group consisting of miR-375, miR-34b, TERC, c-Kit, QPCT, CYP1B1, and PCSK1.

3. The method of claim 1, wherein the gene is miR-375.

4. The method of claim 1, wherein the gene is miR-34b.

5. The method of claim 1, wherein the biological sample comprises skin.

6. The method of claim 5, wherein the biological sample comprises skin epidermis.

7. The method of claim 1, wherein the biological sample comprises melanocytes, melanocytic nevi, keratinocytes, or melanoma cells.

8. The method of claim 7, wherein the melanoma cells are classified as primary in situ, regional metastatic, nodular metastatic, or distant metastatic.

9. The method of claim 1, wherein assessing the level of DNA methylation comprises methylation-specific PCR (MSP).

10. The method of claim 1, wherein assessing the level of DNA methylation comprises a HELP assay, restriction landmark genomic scanning, methylated DNA immunoprecipitation (MeDIP) or highly methylated CpG islands pulled down by methylbinding domain protein MBD2 (Methyl_MBD).

11. The method of claim 3, wherein the CpG island is located from −170 to +58 bp upstream of miR-375 and contains 32 CpG dinucleotides.

12. A method of diagnosing or confirming a pathological stage of melanoma in a subject, said pathological stage being selected from the group consisting of stage I, stage II, stage III, and stage IV, and said method comprising:
(i) assessing the level of DNA methylation in a 5′ upstream CpG island of an epigenetically regulated differentially expressed coding or non-coding gene in a biological sample obtained from the subject; and
(ii) identifying the subject as having stage I melanoma when the CpG island is less than about 20% methylated; identifying the subject as having stage II melanoma when the CpG island is from about 20% to about 60% methylated; identifying the subject as having stage III melanoma when the CpG island is greater than about 60% to about 85% methylated; and identifying the subject as having stage IV melanoma when the CpG island is greater than about 85% methylated.

13. The method of claim 12, wherein the gene is selected from the group consisting of miR-375, miR-34b, TERC, c-Kit, QPCT, CYP1B1, and PCSK1.

14. The method of claim 12, wherein the gene is miR-375.

15. The method of claim 12, wherein the gene is miR-34b.

16. The method of claim 12, wherein the biological sample comprises skin.

17. The method of claim 16, wherein the biological sample comprises skin epidermis.

18. The method of claim 12, wherein the biological sample comprises melanocytes, melanocytic nevi, keratinocytes, or melanoma cells.

19. The method of claim 18, wherein the melanoma cells are classified as primary in situ, regional metastatic, nodular metastatic, or distant metastatic.

20. The method of claim 12, wherein assessing the level of DNA methylation comprises methylation-specific PCR (MSP) or bisulphite DNA sequencing.

21. The method of claim 12, wherein assessing the level of DNA methylation comprises a HELP assay, restriction landmark genomic scanning, or methylated DNA immunoprecipitation (MeDIP).

22. The method of claim 14, wherein the CpG island is located from −170 to +58 bp upstream of miR-375 and contains 32 CpG dinucleotides.

23. A method for diagnosing melanoma in a subject suspected of having melanoma comprising:
(i) assessing the expression level of an epigenetically regulated differentially expressed coding or non-coding gene in a biological sample obtained from the subject; and
(ii) comparing the expression level of the gene in the sample to a reference expression level derived from the expression level of the gene in samples obtained from subjects diagnosed as not having melanoma; and
(iii) identifying the subject as having melanoma when the expression level of the gene in the sample is not greater than the reference expression level or identifying the subject as not having melanoma when the expression level of the gene in the sample is greater than the reference expression level.

24. The method of claim 23, wherein the gene is selected from the group consisting of miR-375, miR-34b, TERC, c-Kit, QPCT, CYP1B1, and PCSK1.

25. The method of claim 23, wherein the gene is miR-375.

26. The method of claim 23, wherein the gene is miR-34b.

27. The method of claim 23, wherein the biological sample comprises skin.

28. The method claim 27, wherein the biological sample comprises skin epidermis.

29. The method claim 23, wherein the biological sample comprises melanocytes, melanocytic nevi, keratinocytes, or melanoma cells.

30. The method of claim 23, wherein the expression level of the gene is assessed by evaluating the amount of the gene's mRNA in the biological sample.

31. The method of claim 30, wherein evaluating the gene comprises array hybridization, wherein the array comprises an immobilized nucleic acid probe that specifically hybridizes the gene, cDNA of the gene, or complements thereof.

32. A method for treating a patient diagnosed as having melanoma comprising administering to the patient an effective amount of a therapeutic agent that increases expression of a gene selected from the group consisting of miR-375, miR-34b, TERC, c-Kit, QPCT, CYP1B1, and PCSK1.

33. The method of claim 32, wherein the gene is miR-375.

34. The method of claim 32, wherein the gene is miR-34b.

35. The method of claim 32, wherein the expression of the gene is increased by at least 10%.

36. The method of claim 32, wherein the expression of the gene is increased by at least 50%.

37. The method of claim 32, wherein the expression of the gene is increased by at least 100%.

38. The method of claim 32, wherein the therapeutic agent decreases CpG island methylation.

39. The method of claim 32, wherein the therapeutic agent is an anti-sense nucleic acid.

40. The method of claim 39, wherein the anti-sense nucleic acid is encoded in a vector.

41. The method of claim 40, wherein the vector is a viral vector.

42. The method of claim 32, wherein the therapeutic agent is contained within a liposome.

43. The method of claim 32, wherein the therapeutic agent is 5AzadC.

44. The method of claim 43, wherein the 5AzadC is administered in conjunction with 4-PBA.

45. A method for determining the invasiveness of melanoma in a subject comprising:
(i) assessing the level of expression of an epigenetically regulated differentially expressed coding or non-coding gene in a biological sample obtained from the subject; and
(ii) determining the invasiveness of the melanoma, wherein a higher expression level of the gene in the sample indicates lower invasiveness and a lower expression level of the gene in the sample indicates greater invasiveness.

46. The method of claim 45, wherein the expression level of the gene is assessed by evaluating the amount of the gene's mRNA in the melanoma sample.

47. The method of claim 45, wherein evaluating the gene's mRNA comprises array hybridization, wherein the array comprises an immobilized nucleic acid probe that specifically hybridizes the gene's mRNA, the gene's cDNA, or complements thereof.

48. The method of claim 45, wherein the gene is selected from the group consisting of miR-375, miR-34b, TERC, c-Kit, QPCT, CYP1B1, and PCSK1.

49. A method for determining the metastatic potential of melanoma in a subject comprising:
(i) assessing the expression level of an epigenetically regulated differentially expressed coding or non-coding gene in a melanoma sample obtained from the subject; and
(ii) determining the metastatic potential of the melanoma, wherein a higher expression level of the gene in the sample indicates a lower metastatic potential and a lower expression level of the gene in the sample indicates a greater metastatic potential.

50. The method of claim 49, wherein the expression level of the gene is assessed by evaluating the amount of the gene's mRNA in the melanoma sample.

51. The method of claim 49, wherein the gene is selected from the group consisting of miR-375, miR-34b, TERC, c-Kit, QPCT, CYP1B1, and PCSK1.

52. The method of claim 49, wherein evaluating the mRNA comprises array hybridization, wherein the array comprises an immobilized nucleic acid probe that specifically hybridizes the gene's mRNA, the gene's cDNA, or complements thereof.

53. A method for determining the prognosis of a patient diagnosed as having melanoma comprising:
(i) assessing the expression level of a gene selected from the group consisting of miR-375, miR-34b, TERC, c-Kit, QPCT, CYP1B1, and PCSK1 in a melanoma sample obtained from the subject; and
(ii) comparing the expression level of the gene in the melanoma sample to the expression level of the gene in a reference sample, wherein a lower expression level of the gene in the melanoma sample relative to the reference sample indicates a poor prognosis.

54. The method of claim 53, wherein the expression level of the gene is assessed by evaluating the amount of the gene's mRNA in the melanoma sample.

55. The method of claim 54, wherein evaluating the gene's mRNA comprises array hybridization, wherein the array comprises an immobilized nucleic acid probe that specifically hybridizes the gene's mRNA, the gene's cDNA, or complements thereof.
